(19) United States
(12) Patent Application Publication
(10) Pub. No.: US 2024/0362782 A1
(43) Pub. Date: Oct. 31, 2024

(54) MEDICAL IMAGE PROCESSING DEVICE, TREATMENT SYSTEM, MEDICAL IMAGE PROCESSING METHOD, AND STORAGE MEDIUM

(71) Applicants: Toshiba Energy Systems & Solutions Corporation, Kawasaki-shi (JP); National Institutes for Quantum Science and Technology, Chiba-shi (JP)

(72) Inventors: Ryusuke HIRAI, Meguro Tokyo (JP); Yukinobu SAKATA, Kawasaki Kanagawa (JP); Akiyuki TANIZAWA, Kawasaki Kanagawa (JP); Kenta UMENE, Fuchu Tokyo (JP); Saori ASAKA, Yokohama Kanagawa (JP); Keiko OKAYA, Setagaya Tokyo (JP); Shinichiro MORI, Chiba-shi (JP)

(73) Assignees: Toshiba Energy Systems & Solutions Corporation, Kawasaki-shi (JP); National Institutes for Quantum Science and Technology, Chiba-shi (JP)

(21) Appl. No.: 18/769,928

(22) Filed: Jul. 11, 2024

Related U.S. Application Data
(63) Continuation of application No. PCT/JP2022/040549, filed on Oct. 28, 2022.

(30) Foreign Application Priority Data
Feb. 8, 2022 (JP) 2022-018143

(51) Int. Cl. G06T 7/00

(52) U.S. Cl. CPC G06T 7/0012; G06T 2207/10121

(57) ABSTRACT

According to an embodiment, a medical image processing device includes a first image acquirer, a second image acquirer, a treatment error acquirer, a difference calculator, and a differential statistical quantity calculator. The first image acquirer acquires a first fluoroscopic image of a patient. The second image acquirer acquires a second fluoroscopic image photographed at a timing different from the first fluoroscopic image. The treatment error acquirer acquires a treatment error occurring when an alignment process is performed or a treatment error occurring in treatment. The difference calculator calculates a difference image between the second fluoroscopic image to which the virtual perturbation is applied at the position of the patient shown therein based on the treatment error and the first fluoroscopic image. The differential statistical quantity calculator calculates a statistical quantity of a difference between the first fluoroscopic image and the second fluoroscopic image based on the difference image.

### CROSS-REFERENCE TO RELATED APPLICATION

This application is based upon and claims the benefit of priority from Japanese Patent Application No. 2022-018143, filed Feb. 8, 2022 and PCT/JP2022/040549, filed Oct. 28, 2022; the entire contents all of which are incorporated herein by reference.

### FIELD

Embodiments of the present invention relate to a medical image processing device, a treatment system, a medical image processing method, and a storage medium.

### BACKGROUND

Radiation treatment is a treatment method of irradiating a tumor (a lesion) within a patient's body with radiation to destroy the tumor. In the radiation treatment, it is necessary to accurately radiate the radiation to the position of the tumor because normal tissues within the patient's body that are irradiated with the radiation may be affected. Thus, when the radiation treatment is performed, computed tomography (CT) is first performed, for example, in advance in a treatment planning phase, and the position of the tumor within the patient's body is three-dimensionally ascertained. A radiation irradiation direction and a radiation irradiation intensity are planned on the basis of the ascertained position of the tumor. Thereafter, the position of the patient in a treatment phase is aligned with the position of the patient planned in the treatment planning phase and the tumor is irradiated with radiation in accordance with an irradiation direction or an irradiation intensity planned in the treatment planning phase.

In the position alignment of the patient in the treatment phase, image collation between a fluoroscopic image of the inside of the patient's body captured in a state in which the patient is laid on the patient table immediately before the start of treatment and a digitally reconstructed radiograph (DRR) image in which the fluoroscopic image is virtually reconstructed from a three-dimensional CT image captured at the time of the treatment planning is performed and the deviation in the position of the patient between images is obtained. The patient table is moved on the basis of the obtained deviation. Thereby, the position of a tumor, bone, or the like within the patient's body is aligned with that planned in the treatment plan.

The position deviation of the patient is obtained by seeking the position in the CT image so that the DRR image most similar to the fluoroscopic image is reconstructed. In the related art, many methods of automatically seeking the position of a patient using a computer have been proposed. However, a user (a doctor or the like) finally confirms an automated search result by comparing the fluoroscopic image with the DRR image in the related art. For example, in the conventional method, a process in which the fluoroscopic image and the DRR image are made semi-transparent and superimposed so that the user can visually confirm that the contours of the edge portions of the bone match is performed. However, this confirmation method is not a method of quantifying and expressing a degree of matching of the patient's position and the like. For this reason, there is a possibility that an effect of treatment that has been performed may differ depending on an influence of the user's ability to perform the confirmation or the like in a method in which the user performs visual confirmation.

Meanwhile, it may be difficult to visually confirm the position of a tumor shown in a fluoroscopic image. This is because a tumor is more transparent to X-rays than a bone and the like and therefore is not clearly shown in a fluoroscopic image. Therefore, when treatment is performed, a CT image has been captured instead of a fluoroscopic image to confirm the position of the tumor in the recent years. In this case, the deviation in the patient's position is obtained by performing an image collation process between the CT image captured at the time of treatment planning and the CT image captured in the treatment phase, i.e., by performing an image collation process between the CT images.

In the image collation process between CT images, while the position of one CT image is shifted, the position of an image most similar to the other CT image is obtained. In an image collation process between CT images, for example, a method of comparing pixel values of two CT images and searching for the position where a difference is the smallest and the like can be considered. Furthermore, as another example in which an image collation process is performed between CT images, for example, there is a method of calculating an amount of energy loss when radiation passes through a human body (CT data) using CT images and obtaining the position where the amount of energy loss matches. Because the degree of matching of the amount of energy loss in this case is quantitative, there is no need to enforce the determination of the user and it may be possible to mechanically determine whether or not the alignment of the patient is successful.

On the other hand, if CT images can be captured during treatment, it is also possible to replan the treatment plan to accommodate changes in a state of the patient (e.g., changes in a posture of the patient or the like) over time as the treatment progresses. For example, it is possible to change the treatment plan on the basis of a difference between two CT images. In this case, it is necessary to present a portion where there is a difference between the two CT images to the user. For example, an amount of change in the position of the patient that matches through the alignment is presented to the user. However, in the alignment of the patient in the treatment phase, because the position of the patient is adjusted by physically moving the patient table with the patient lying down, for example, there is a possibility that an error, which does not appear in computational alignment based on image data as in an image collation process between CT images, such as a movement error of the patient table, may occur.

### BRIEF DESCRIPTION OF THE DRAWINGS

FIG. 1 is a block diagram showing a schematic configuration of a treatment system including a medical image processing device of a first embodiment.

FIG. 2 is a block diagram showing a schematic configuration of the medical image processing device.

FIG. 3 is a flowchart showing a flow of a process of outputting a differential statistical quantity in the medical image processing device.

FIG. 4 is a block diagram showing a schematic configuration of a medical image processing device of a second embodiment.

FIG. 5 is a flowchart showing a flow of a process of determining whether or not it is necessary to adjust the position of a patient in the medical image processing device.

FIG. 6 is a block diagram showing a schematic configuration of a medical image processing device of a third embodiment.

FIG. 7 is a diagram showing an example of presentation data generated by a presentation data processor provided in the medical image processing device.

FIG. 8 is a diagram showing an example of a display screen in which the presentation data processor provided in the medical image processing device causes a display device to display the presentation data.

FIG. 9 is a diagram showing an example of another display screen in which the presentation data processor provided in the medical image processing device causes the display device to display the presentation data.

### DETAILED DESCRIPTION

According to an aspect of the present embodiment, a medical image processing device includes a first image acquirer, a second image acquirer, a treatment error acquirer, a difference calculator, and a differential statistical quantity calculator. The first image acquirer acquires a first fluoroscopic image obtained by photographing the inside of a body of a patient. The second image acquirer acquires a second fluoroscopic image of the inside of the body of the patient photographed at a timing different from that of the first fluoroscopic image. The treatment error acquirer acquires a treatment error occurring when an alignment process of aligning the position of the patient shown in the second fluoroscopic image with the position of the patient shown in the first fluoroscopic image is performed on the basis of the first fluoroscopic image and the second fluoroscopic image or a treatment error occurring in treatment. The difference calculator applies a virtual perturbation to the position of the patient shown in the second fluoroscopic image on the basis of the treatment error and calculates a difference image between the second fluoroscopic image to which the perturbation is applied and the first fluoroscopic image. The differential statistical quantity calculator calculates a statistical quantity of a difference between the first fluoroscopic image and the second fluoroscopic image to which the perturbation is applied on the basis of the difference image.

Hereinafter, a medical image processing device, a treatment system, a medical image processing method, and a storage medium according to embodiments will be described with reference to the drawings.

### First Embodiment

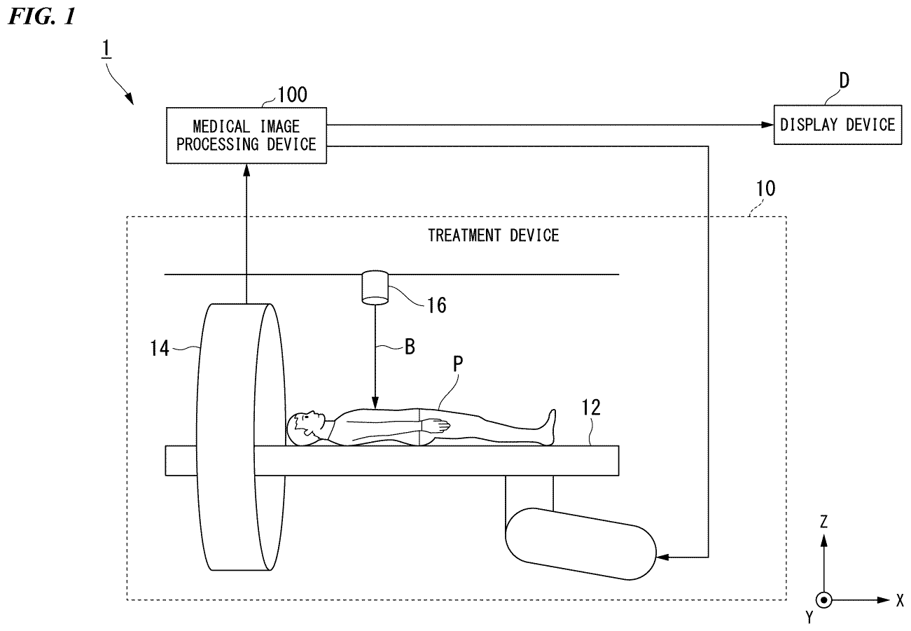

FIG. 1 is a block diagram showing a schematic configuration of a treatment system including a medical image processing device of a first embodiment. A treatment system 1 includes, for example, a treatment device 10 and a medical image processing device 100. The treatment device 10 includes, for example, a patient table 12, a computed tomography (CT) device 14 (hereinafter referred to as a “CT photography device 14”), and a treatment beam irradiation gate 16.

The patient table 12 is a movable treatment table to which a subject (a patient) P who is to be treated with radiation is fixed in a lying state, for example, by a fixing tool or the like. Under control of the medical image processing device 100, the patient table 12 moves into the ring-shaped CT photography device 14 having an opening in a state in which the patient P is fixed thereto. The medical image processing device 100 outputs a movement control signal for controlling a translation mechanism and a rotation mechanism provided on the patient table 12 to change a direction of irradiation of a treatment beam B to the patient P fixed to the patient table 12. The translation mechanism can drive the patient table 12 in three axis directions and the rotation mechanism can drive the patient table 12 around three axes. Thus, the medical image processing device 100 controls, for example, the translation mechanism and the rotation mechanism of the patient table 12 so that the patient table 12 moves with six degrees of freedom. The degrees of freedom with which the medical image processing device 100 controls the patient table 12 may not be six degrees of freedom and may be fewer than six degrees of freedom (for example, four degrees of freedom or the like) or more than six degrees of freedom (for example, eight degrees of freedom or the like).

The CT photography device 14 is an imaging device for performing three-dimensional computed tomography. The CT photography device 14 has a plurality of radiation sources arranged inside of a ring-shaped opening and radiates radiation for fluoroscopy inside of the body of the patient P from each radiation source. That is, the CT photography device 14 irradiates the patient P with radiation from a plurality of positions near the patient. Radiation radiated from each radiation source in the CT photography device 14 is, for example, X-rays. The CT photography device 14 detects the radiation radiated from the corresponding radiation source and received after passing through the inside of the body of the patient P using a plurality of radiation detectors arranged inside of the ring-shaped opening. The CT photography device 14 generates a CT image of the inside of the patient P on the basis of a magnitude of radiation energy detected by each radiation detector. A CT image of the patient P generated by the CT photography device 14 is a three-dimensional digital image in which the magnitude of the radiation energy is represented by a digital value. The CT photography device 14 outputs the generated CT image to the medical image processing device 100. For example, a photography controller (not shown) controls the three-dimensional photography of the inside of the body of the patient P by the CT photography device 14, i.e., the irradiation of radiation from each radiation source and the generation of a CT image based on the radiation detected by each radiation detector. The CT photography device 14 is an example of an “imaging device.”

The treatment beam irradiation gate 16 radiates radiation as a treatment beam B for destroying a tumor (a lesion), which is a site to be treated inside of the patient P's body. The treatment beam B is, for example, X-rays, y-rays, an electron beam, a proton beam, a neutron beam, a heavy particle beam, or the like. The treatment beam B is linearly radiated from the treatment beam irradiation gate 16 to the patient P (more specifically, the tumor inside of the body of the patient P). Irradiation of the treatment beam B at the treatment beam irradiation gate 16 is controlled by, for example, a treatment beam irradiation controller (not shown). The treatment beam irradiation gate 16 is an example of an “irradiator.”

In a treatment room where the treatment system 1 is installed, three-dimensional coordinates of a reference position as shown in FIG. 1 are preset. In the treatment room where the treatment beam B is radiated to the patient P, an installation position of the treatment beam irradiation gate 16, a direction in which the treatment beam B is radiated (an irradiation direction), an installation position of the patient table 12, an installation position of the CT photography device 14, a photography position of a CT image obtained by photographing the inside of the patient P, or the like is ascertained in accordance with the preset three-dimensional coordinates of the reference position. In the following description, the three-dimensional coordinate system of the preset reference position in the treatment room is defined as a “room coordinate system.” In the following description, it is assumed that a “position” is a position of (three-dimensional) coordinates in the three axis directions based on the translation mechanism of the patient table 12 indicated in accordance with the room coordinate system and an “orientation” is an orientation of a rotational angle around three axes based on the rotation mechanism provided in the patient table 12 represented by the room coordinate system. For example, the position of the patient table 12 is the position of a prescribed point included in the patient table 12 represented by three-dimensional coordinates and the orientation of the patient table 12 is represented by a rotational angle of the patient table 12 in yaw, roll, and pitch.

In radiation treatment, a treatment plan is created in a situation in which the treatment room is simulated. That is, in the radiation treatment, an irradiation direction, an intensity, and the like when the patient P is irradiated with the treatment beam B are planned by simulating a state in which the patient P is placed on the patient table 12 in the treatment room. Thus, information such as parameters indicating the position and orientation of the patient table 12 within the treatment room is added to the CT image in the phase of treatment planning (the treatment planning phase). The same is also true for CT images captured immediately before radiation treatment and CT images captured during previous radiation treatment. In other words, parameters indicating the position and orientation of the patient table 12 at the time of photography are assigned to a CT image obtained by photographing the inside of the body of the patient P with the CT photography device 14.

Although the configuration of the treatment device 10 including the CT photography device 14 and one treatment beam irradiation gate 16 that has been fixed is shown in FIG. 1, the configuration of the treatment device 10 is not limited to the above-described configuration. For example, instead of the CT photography device 14, the treatment device 10 may have a configuration including a photography device for generating an image obtained by three-dimensionally photographing the inside of the body of the patient P such as a CT photography device in which a set of a radiation source and a radiation detector rotates inside of a ring-shaped opening, a cone-beam (CB) CT device, a magnetic resonance imaging (MRI) device, or an ultrasonic diagnostic device. For example, the treatment device 10 may have a configuration including a plurality of treatment beam irradiation gates such as a configuration further including a treatment beam irradiation gate for irradiating the patient P with the treatment beam from the horizontal direction. For example, the treatment device 10 may have a configuration in which the patient P is irradiated with treatment beams from various directions when the one treatment beam irradiation gate 16 shown in FIG. 1 performs the rotation around the patient P such as the rotation of 360 degrees about a rotation axis of a horizontal direction X shown in FIG. 1. For example, instead of the CT photography device 14, the treatment device 10 includes one or more imaging devices each including a set of a radiation source and a radiation detector and may be configured to photograph the inside of the body of the patient P from various directions when the imaging devices rotate 360 degrees about the rotation axis of the horizontal direction X shown in FIG. 1. This configuration is referred to as a rotating gantry type treatment device. In this case, for example, one treatment beam irradiation gate 16 shown in FIG. 1 may be configured to rotate around the same rotation axis simultaneously with the imaging device.

The medical image processing device 100 performs a process for aligning the position of the patient P when radiation treatment is performed, on the basis of a CT image output by the CT photography device 14. More specifically, the medical image processing device 100 performs, for example, a process for aligning the position of a tumor or tissues located inside of the body of the patient P on the basis of a CT image of the patient P captured before radiation treatment is performed in a treatment planning phase or the like and a current CT image of the patient P captured by the CT photography device 14 in the phase of treatment (the treatment phase) in which radiation treatment is performed. The medical image processing device 100 outputs the movement control signal and moves the patient table 12 so that the irradiation direction of the treatment beam B radiated from the treatment beam irradiation gate 16 is aligned with the direction set in the treatment planning phase. That is, the medical image processing device 100 moves the patient P in a direction in which the treatment beam B is appropriately applied to the tumor or tissues to be treated in the radiation treatment in accordance with the movement control signal.

The medical image processing device 100 and the CT photography device 14 provided in the treatment device 10 may be connected by wire or may be wirelessly connected by, for example, a local area network (LAN), a wide area network (WAN), or the like.

Furthermore, the medical image processing device 100 presents information indicating a result of a process for aligning the position of the patient P (hereinafter referred to as an “alignment process”) (or information indicating that the process is ongoing) to a radiation treatment practitioner such as a doctor, i.e., a user of the treatment system 1. In FIG. 1, the medical image processing device 100 has, for example, a configuration in which the result of the alignment process is presented to a person using the result of the alignment process (hereinafter referred to as a “user”) by displaying an image or information on a display device D such as a liquid crystal display (LCD), an organic electroluminescence (EL) display, and a micro light emitting diode (LED) display. The display device D may be, for example, a display device (so-called personal computer (PC) monitor) connected to a PC or a display device provided in a terminal device (digital device) such as a tablet terminal or a smartphone. Thereby, the user can confirm the result of the alignment process displayed on the display device D and determine whether to end the alignment process, i.e., whether to start the radiation treatment or whether to perform the alignment process again. In addition to information indicating the result of the alignment process, for example, the medical image processing device 100 may present a CT image of the treatment planning phase or a CT image of the treatment phase to the user by displaying the CT image of the treatment planning phase or the CT image of the treatment phase on the display device D. However, because the CT image is a three-dimensional image, the image cannot be directly displayed on the display device D that performs a two-dimensional display process. For this reason, the medical image processing device 100 generates one or more cross-sectional images corresponding to each of the CT image in the treatment planning phase and the CT image in the treatment phase and displays the generated cross-sectional images on the display device D. At this time, in order to make it easier for the user to visually compare the CT images, the medical image processing device 100 may display a difference image obtained by taking a difference between cross-sectional images or may display a color-coded color map in accordance with a magnitude of a difference value between cross-sectional images.

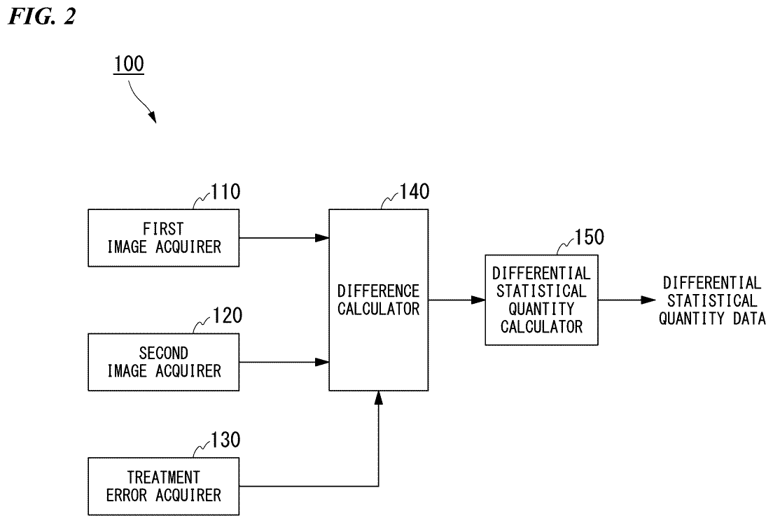

The medical image processing device 100 of the first embodiment will be described below. FIG. 2 is a block diagram showing a schematic configuration of the medical image processing device 100. The medical image processing device 100 includes, for example, a first image acquirer 110, a second image acquirer 120, a treatment error acquirer 130, a difference calculator 140, and a differential statistical quantity calculator 150.

Some or all of the constituent elements provided in the medical image processing device 100 are implemented by, for example, a hardware processor such as a central processing unit (CPU) executing a program (software). Also, some or all of these constituent elements may be implemented by hardware (including a circuit unit; circuitry) such as a large-scale integration (LSI) circuit, an application-specific integrated circuit (ASIC), a field-programmable gate array (FPGA), or a graphics processing unit (GPU) or may be implemented by software and hardware in cooperation. Also, some or all of the functions of the constituent elements may be implemented by a dedicated LSI circuit. The program may be pre-stored in a storage device (a storage device including a non-transitory storage medium) such as a read-only memory (ROM), a random-access memory (RAM), a hard disk drive (HDD), or a flash memory provided in the medical image processing device 100 or may be stored in a removable storage medium (a non-transitory storage medium) such as a DVD or a CD-ROM and installed in the HDD or the flash memory provided in the medical image processing device 100 when the storage medium is mounted in a drive device provided in the medical image processing device 100. Also, the program may be downloaded in advance from another computer device via the network and installed in the HDD or the flash memory provided in the medical image processing device 100.

The first image acquirer 110 acquires a first fluoroscopic image related to the patient P before treatment and parameters indicating the position and an orientation when the first fluoroscopic image has been captured. The first fluoroscopic image is a three-dimensional CT image representing a three-dimensional shape inside of the body of the patient P captured by, for example, the CT photography device 14 in the treatment planning phase when radiation treatment is performed. The first fluoroscopic image is used to decide on a direction (a path including an inclination, a distance, and the like) and an intensity of the treatment beam B to be radiated to the patient P in radiation treatment. The decided direction (irradiation direction) and intensity of the treatment beam B are set in the first fluoroscopic image. The first fluoroscopic image is captured in a state in which the patient P is fixed to the patient table 12 and the uniform position and orientation of the patient P (hereinafter referred to as a “body posture”) are maintained. A parameter indicating the body posture of the patient P when the first fluoroscopic image has been captured may be a position or an orientation (a photography direction or photography magnification) of the CT photography device 14 when the first fluoroscopic image has been captured or may be a setting value set in the translation mechanism and the rotation mechanism provided in the patient table 12, for example, so that the position and the orientation of the patient table 12 when the first fluoroscopic image has been captured, i.e., the body posture of the patient P, are maintained uniformly. The first image acquirer 110 outputs the acquired first fluoroscopic image and parameters to the difference calculator 140. If the first fluoroscopic image is an image captured before radiation treatment is performed, for example, it may be an image captured immediately before treatment is performed in the treatment room or an image captured during previous radiation treatment. The first image acquirer 110 may have an interface for connecting to the CT photography device 14 provided in the treatment device 10.

The second image acquirer 120 acquires a second fluoroscopic image related to the patient P immediately before radiation treatment starts and parameters indicating the position and the orientation when the second fluoroscopic image has been captured. The second fluoroscopic image is, for example, a three-dimensional CT image representing the three-dimensional shape inside of the body of the patient P captured by the CT photography device 14 so that the body posture of the patient P is aligned when the treatment beam B is radiated in the radiation treatment. That is, the second fluoroscopic image is an image captured by the CT photography device 14 in a state in which the treatment beam B is not radiated from the treatment beam irradiation gate 16. In other words, the second fluoroscopic image is a CT image captured at a timing different from a timing when the first fluoroscopic image has been captured. In this case, photography timings of the first fluoroscopic image and the second fluoroscopic image are different from each other, but image capturing methods for the first fluoroscopic image and the second fluoroscopic image are similar to each other. Thus, the second fluoroscopic image is captured in a body posture similar to the body posture when the first fluoroscopic image has been captured. A parameter indicating the body posture of the patient P when the second fluoroscopic image has been captured may be a position or an orientation (a photography direction or photography magnification) of the CT photography device 14 when the second fluoroscopic image has been captured or may be a setting value set in the translation mechanism and the rotation mechanism provided in the patient table 12, for example, so that the position and the orientation of the patient table 12, i.e., the body posture of the patient P, when the second fluoroscopic image has been captured are close to a body posture when the first fluoroscopic image has been captured. The second image acquirer 120 outputs the acquired second fluoroscopic image and parameters to the difference calculator 140. The second image acquirer 120 may have an interface for connecting to the CT photography device 14 provided in the treatment device 10. This interface may be the same as the interface provided in the first image acquirer 110.

The first fluoroscopic image and the second fluoroscopic image are not limited to CT images captured by the CT photography device 14 and may be three-dimensional images captured by an imaging device different from the CT photography device 14, for example, such as a CBCT device, an MRI device, or an ultrasonic diagnostic device. For example, the first fluoroscopic image may be a CT image and the second fluoroscopic image may be a three-dimensional image captured by an MRI device. In contrast, the first fluoroscopic image may be a three-dimensional image captured by an MRI device, and the second fluoroscopic image may be a CT image. The first fluoroscopic image and the second fluoroscopic image are not limited to a three-dimensional image, and may be a four-dimensional image such as, for example, a CT image captured from a moving image. The first and second fluoroscopic images may be two-dimensional X-ray images captured from one or more directions.

As described above, both the first fluoroscopic image and the second fluoroscopic image are three-dimensional CT images captured at different timings. Also, a body posture of the patient P when the second fluoroscopic image is captured is kept close to a body posture when the first fluoroscopic image has been captured. However, it is difficult to capture the second fluoroscopic image at the body posture of the patient P that is exactly the same as that when the first fluoroscopic image has been captured. That is, it is difficult to suppress a change in a state of the inside of the body of the patient P or to fix the body at the same body posture even if a fixing tool is used. For this reason, even if the body posture of the patient P shown in the first fluoroscopic image and the body posture of the patient P shown in the second fluoroscopic image are virtually equally arranged in a predetermined three-dimensional space, a slight deviation (for example, several millimeters (mm)) occurs and it is difficult to reproduce the body posture of the patient P when the first fluoroscopic image has been captured only by capturing the second fluoroscopic image. The predetermined three-dimensional space is a space of a room coordinate system preset in a treatment room. Therefore, in the medical image processing device 100, an approximate image for the first fluoroscopic image is calculated in the alignment process and amounts of deviation between the positions and orientations of the first fluoroscopic image and the second fluoroscopic image are further calculated and an amount of movement of the patient table 12 for aligning the positions of the body posture of the patient P shown in the first fluoroscopic image and the body posture of the patient P shown in the second fluoroscopic image is decided. That is, the medical image processing device 100 decides the amount of movement of the patient table 12 for reproducing the body posture of the patient P when the first fluoroscopic image has been captured in the alignment process. At this time, the medical image processing device 100 may perform an alignment process using a fluoroscopic image having a smaller number of pixels between the first fluoroscopic image and the second fluoroscopic image as a reference. In this case, the time required for the alignment process can be shortened.

Here, an example of the alignment process in the medical image processing device 100 will be described.

First, the treatment plan performed before the alignment process of the medical image processing device 100 is performed will be described. In the treatment plan, energy of treatment beams B (radiation) radiated to the patient P, an irradiation direction, a shape of the irradiation range, and a distribution of doses when the treatment beams B are divided and radiated a plurality of times, and the like are defined. More specifically, first, a planner (a doctor or the like) of the treatment plan first designates a boundary between a region of a tumor (a lesion) and a normal tissue region, a boundary between the tumor and a vital organ near the tumor, and the like with respect to the first fluoroscopic image that has been captured (for example, a CT image captured by the CT photography device 14) in the treatment planning phase. In the treatment plan, a direction in which the treatment beam B is radiated (a path through which the treatment beam B passes), an intensity, or the like is decided on the basis of a depth from a body surface of the patient P to the position of a tumor or the size of the tumor calculated from information about the tumor designated by the planner (a doctor or the like) of the treatment plan.

The designation of the boundary between the tumor region and the normal tissue region corresponds to the designation of the position and a volume of the tumor. The volume of this tumor is referred to as a gross tumor volume (GTV), a clinical target volume (CTV), an internal target volume (ITV), a planning target volume (PTV), or the like. The GTV is a volume of the tumor capable of being visually confirmed from the image and is a volume required to be irradiated with a sufficient dose of the treatment beams B in radiation treatment. The CTV is a volume including the GTV and a latent tumor to be treated. The ITV is a volume obtained by adding a prescribed margin to the CTV in consideration of the movement of the CTV due to predicted physiological movement of the patient P and the like. The PTV is a volume obtained by adding a margin to the ITV in consideration of an error in position alignment of the patient P performed when treatment is performed. The relationship of the following Expression (1) is established between these volumes.

$$\begin{matrix}[ Math . 1 ] & \\ GTV ∈ CTV ∈ ITV ∈ PTV & ( 1 )\end{matrix}$$

On the other hand, a volume of a vital organ located near the tumor, which is highly sensitive to radiation and strongly affected by the dose of irradiated radiation, is referred to as an organ at risk (OAR). A planning organ at risk volume (PRV) is designated as a volume obtained by adding a prescribed margin to the OAR. The PRV is designated by adding a volume (a region) to which the radiation is radiated as a margin while avoiding the OAR which is not desired to be destroyed by the radiation. The relationship of the following Expression (2) is established between these volumes.

$$\begin{matrix}[ Math . 2 ] & \\ OAR ∈ PRV & ( 2 )\end{matrix}$$

In the treatment planning phase, the direction (path) or intensity of the treatment beam B (radiation) radiated to the patient P is decided on the basis of the margin in consideration of an error that may occur in actual treatment.

Subsequently, when the medical image processing device 100 performs the alignment process in the treatment phase of radiation treatment, the first image acquirer 110 first acquires a first fluoroscopic image and parameters indicating the position and orientation of the first fluoroscopic image. The second image acquirer 120 acquires a second fluoroscopic image of the patient P immediately before treatment starts and parameters indicating the position and orientation of the second fluoroscopic image. The medical image processing device 100 performs the alignment process with information about a direction within the treatment room (hereinafter referred to as “direction information”). The direction information is information expressed in a preset room coordinate system. The direction information includes, for example, information indicating an irradiation direction of a treatment beam B and information indicating a movement direction of the patient table 12.

Information indicating the irradiation direction of the treatment beam B is information indicating a direction in which the treatment beam irradiation gate 16 radiates the treatment beam B to the patient P in the treatment room. The treatment device 10 may have a configuration in which the treatment beam irradiation gate 16 is fixed as shown in FIG. 1, but may also have, for example, a configuration in which the treatment beam B can be radiated in a vertical direction and a horizontal direction as described above or a configuration in which the treatment beam irradiation gate 16 can be rotated simultaneously with the imaging device around the same rotation axis and can radiate treatment beams B in various directions. Furthermore, the treatment beam B may be radiated to a region (range) of the tumor present inside the body of the patient P by scanning (raster scanning) a beam of radiation or radiating the beam within a planar range of a predetermined size. That is, the irradiation direction of the treatment beam B may have a plurality of paths when the treatment beam B is actually irradiated to the tumor inside the body of patient P. In these cases, the medical image processing device 100 acquires all irradiation directions (including a plurality of paths) in which the treatment beam B can be radiated in the treatment room as information indicating the irradiation direction of the treatment beam B.

Information indicating the movement direction of the patient table 12 is information indicating a direction in which the fixed patient P can be moved when the treatment beam B is radiated with the patient table 12 installed in the treatment room. The information indicating the movement direction of the patient table 12 also includes information indicating an angle at which the body posture of the patient P can be changed by the patient table 12. For example, the patient table 12 can move the position and body posture with six degrees of freedom using the translation mechanism and the rotation mechanism as described above. For this reason, the information indicating the movement direction of the patient table 12 may be information of directions of the six degrees of freedom in the patient table 12. The information indicating the movement direction of the patient table 12 may be information indicating ranges of setting values that can be set in the translation mechanism and the rotation mechanism. As described above, when the patient table 12 moves with fewer degrees of freedom than six degrees of freedom (for example, four degrees of freedom or the like), the medical image processing device 100 acquires information corresponding to the degrees of freedom with which the patient table 12 moves. A case where the movement of the patient table 12 conforms to a unique coordinate system different from the room coordinate system set in advance in the treatment room is also conceivable. In this case, the medical image processing device 100 may acquire information of the movement direction in the unique coordinate system to which the patient table 12 conforms as information indicating the movement direction of the patient table 12.

The medical image processing device 100 performs an alignment process using the acquired information indicating the irradiation direction of the treatment beam B and the information indicating the movement direction of the patient table 12. The medical image processing device 100 outputs a movement control signal corresponding to the result of the alignment process to the treatment device 10. Thereby, the treatment device 10 moves the patient table 12 so that the current body posture of the patient P is close to the body posture of the patient P in the treatment planning phase in accordance with the movement control signal output by the medical image processing device 100.

The treatment error acquirer 130 acquires an error in radiation treatment not appearing in the treatment planning phase (hereinafter referred to as a “treatment error”). The treatment error acquirer 130 outputs the acquired treatment error to the difference calculator 140. The treatment error is an error considered to occur when the radiation treatment is performed.

The treatment error is, for example, a movement error of the patient table 12 that is assumed (estimated) to occur when the position and orientation of the patient table 12 are moved in accordance with the movement control signal. The movement error is, for example, a small error in mechanical control that is likely to occur when the translation mechanism and the rotation mechanism provided in the patient table 12 move the patient table 12. For example, when the patient table 12 is a robot arm type patient table device attached to the tip of the arm, the patient table 12 can be moved to any coordinate position in the room coordinate system in the treatment room by moving an angle of a joint of the robot arm or the position of a base thereof according to the movement control signal. However, in this case, likewise, there is also a possibility that a small error will occur in the mechanical control because a process of controlling the robot arm is mechanical control. The movement error of the patient table 12 can be measured, for example, by measuring the coordinates of the patient table 12 using a distance sensor fixed in the treatment room and capable of highly accurate measurement, such that a difference from the coordinates indicated in the movement control signal can be obtained and this difference can be measured as the movement error of the patient table 12. However, when it is difficult to perform highly accurate measurement with the distance sensor due to time constraints every time radiation treatment is performed, for example, a movement error when the current radiation treatment is performed may be determined on the basis of a movement error distribution measured when a periodic inspection of the treatment system 1 is performed or the like. The distribution of this movement error, for example, may be provided separately for each of the three axes in a translational direction of the patient table 12 and the three axes in a rotational direction, i.e., six axes.

The treatment error is, for example, a deviation assumed (estimated) between the irradiation of the treatment beam B planned in the treatment planning phase and the irradiation of the treatment beam B in the treatment phase. A deviation occurring at the time of irradiation of the treatment beam B is, for example, a deviation (error) of an irradiation position including the direction of the treatment beam B to be radiated to the patient P (i.e., a path including an inclination or distance of the treatment beam B to be radiated and the like) or a deviation (error) of an intensity. As factors of a deviation occurring when such a treatment beam B is radiated, for example, weather conditions such as a temperature, an atmospheric pressure, and a season, a fluctuation over time in a time period of day or the like, and the like are considered to be various factors that affect the irradiation system of the treatment beam B. When it is difficult to measure the deviation (error) of the irradiation position of the treatment beam B every time radiation treatment is performed, for example, a magnitude of the error may be determined on the basis of an experience value of the user (a doctor or the like) of the treatment system 1.

The treatment error is, for example, an installation error of a fixing tool used when the patient P is fixed to the patient table 12, wherein the installation error is assumed (estimated) to be different between the treatment planning phase and the treatment phase. The installation error is an error that is likely to occur because a gap between the patient P and the fixing tool differs between the treatment planning phase and the treatment phase due to a factor such as, for example, a change in a body shape in accordance with an increase or decrease in the weight of the patient P. The magnitude of the installation error, for example, may be determined in advance in accordance with the weight of the patient P or a value of a body surface of the patient P such as chest circumference or abdominal circumference.

The treatment error is, for example, an error related to a state of the inside of the body of the patient P assumed (estimated) to occur due to a change in the body of the patient P over time. Factors of an error related to the state of the inside of the body of patient P may be considered as factors due to physiological activities such as, for example, the location of intestinal gases, pulsation, respiration, swelling, and a blood flow rate. In relation to the error related to the state of the inside of the body of the patient P, for example, the magnitude of the error may be determined on the basis of these numerical values measured for the patient P during radiation treatment.

This treatment error, for example, may be assumed (estimated) by a user (such as a doctor) and input by manipulating an input device such as a user interface (not shown) provided in the medical image processing device 100. The user interface (input device) is, for example, an input device such as a keyboard, a pointing device such as a mouse or a pen-type stylus, and manipulation devices such as buttons and switches. The user interface may include a pressure sensor as an input device and may be configured as a touch panel combined with a display device D. In this case, the user inputs the treatment error by performing manipulations of various types of touches (taps, flicks, and the like) on the image displayed on the display device D. For example, the user inputs information of the treatment error within an allowable range in consideration of various conditions such as a site of the patient P to be subjected to radiation treatment, whether there is a location where the treatment beam B should not be radiated in the vicinity of a place where the treatment beam B is radiated, and the depth at which the treatment beam B is radiated. The treatment error acquirer 130 acquires the treatment error input by the user and outputs the acquired treatment error to the difference calculator 140.

The difference calculator 140 calculates (generates) a difference image between a first fluoroscopic image and a second fluoroscopic image while virtually changing parameters of the second fluoroscopic image (the position and orientation when the second fluoroscopic image has been captured) in accordance with a treatment error in the radiation treatment on the basis of the first fluoroscopic image and parameters output by the first image acquirer 110, the second fluoroscopic image and parameters output by the second image acquirer 120, and the treatment error output by the treatment error acquirer 130. That is, the difference calculator 140 calculates a difference image in which the body posture of the patient P is virtually shifted in consideration of the treatment error in the radiation treatment not appearing in the treatment planning phase. More specifically, the difference calculator 140 calculates a difference image according to a difference between the first fluoroscopic image and the second fluoroscopic image in which a virtual perturbation is applied to the body posture of the patient P (hereinafter, a second fluoroscopic image to which a perturbation is applied is referred to as a “2P^{th} fluoroscopic image” to distinguish it from a second fluoroscopic image before the perturbation is applied) by applying a virtual perturbation to the body posture of the patient P shown in the second fluoroscopic image after the alignment process is performed, i.e., by virtually shifting the body posture of the patient P, on the basis of the treatment error. The size (the number of pixels) of the difference image calculated by the difference calculator 140 may be the same as or different from a size (the number of pixels) of the first fluoroscopic image or the second fluoroscopic image after the alignment process. For example, the difference calculator 140 may calculate a difference image having a size (the number of pixels) corresponding to a range in which the differential statistical quantity calculator 150 to be described below calculates the differential statistical quantity.

The difference calculator 140 may calculate a difference image having a difference from the first fluoroscopic image each time, while changing the virtual perturbations applied to the body posture of the patient P shown in the second fluoroscopic image after the alignment process is performed while sequentially making changes to different perturbations, i.e., while sequentially shifting the body posture of the patient P by a different amount of deviation, on the basis of the treatment error. That is, the difference calculator 140 may apply a plurality of perturbations to the body posture of the patient P shown in the second fluoroscopic image after the alignment process and calculate difference images equal in number to the number of times the perturbations are applied. The number of times that the difference calculator 140 calculates the difference image, i.e., the number of times that virtual perturbations are applied to the body posture of the patient P, may be, for example, the predetermined number of times corresponding to the processing capability of the difference calculator 140 or the medical image processing device 100 or may be the number of times designated by a user (for example, designated by the user manipulating the user interface (not shown)).

The calculation of the difference image in the difference calculator 140, for example, is performed by obtaining a pixel value (CT value) difference between pixels (voxels) at the same position in the first fluoroscopic image and the 2P^{th} fluoroscopic image to which virtual perturbations are applied after the alignment process. Meanwhile, for example, when the first fluoroscopic image is a three-dimensional CT image and the second fluoroscopic image is a two-dimensional X-ray image, the dimensions of the two fluoroscopic images from which the difference image is calculated may be considered to be different. In this case, the difference calculator 140 converts the three-dimensional CT image into a DRR image, thereby aligning the dimensions of the first fluoroscopic image and the second fluoroscopic image after the alignment process, and then applies virtual perturbations to calculate a difference image. The calculation of the difference image in the difference calculator 140 in this case is, for example, performed by obtaining a pixel value difference between pixels at the same position in the first fluoroscopic image and the 2P^{th} fluoroscopic image. The difference calculator 140 may calculate the difference image by applying virtual perturbation to each of the first fluoroscopic image and the second fluoroscopic image after the alignment process after performing various types of image processing such as a smoothing process for suppressing noise, a conversion process such as an edge enhancement process, and a conversion process such as a conversion process for converting the pixel value in a gradient direction and obtaining a pixel value difference between the fluoroscopic images.

The difference image is an image calculated (generated) by focusing on a process of radiating treatment beams B to the tumor or tissue present in the body of the patient P with the dose of the treatment beams B determined in the treatment planning phase. For example, when the second fluoroscopic image is a CT image, the difference calculator 140 generates the difference image by calculating the dose of the treatment beams B to be radiated as in the treatment planning phase and calculating a dose difference between the 2P^{th} fluoroscopic image and the first fluoroscopic image for each pixel. Instead of the dose of the treatment beams B, the difference calculator 140 may generate a difference image by converting each of the first fluoroscopic image and the 2P^{th} fluoroscopic image into an amount of energy attenuation of the treatment beam B in the tumor and calculating a difference. The amount of energy attenuation of the treatment beam B can be obtained, for example, by integrating pixel values (CT values) of pixels (voxels) located on a path through which the radiated treatment beam B passes. The amount of energy attenuation of the treatment beam B, for example, may be converted into a water equivalent thickness. The water equivalent thickness is a value obtained by expressing the amount of energy attenuation of the treatment beam B, which differs according to each tissue (substance), as the thickness of water, which is the same substance, and can be converted on the basis of the CT value. For example, when the CT value is a value indicating bone, because the amount of energy attenuation when the treatment beam B passes through the bone is large, the water equivalent thickness has a large value. For example, when the CT value is a value indicating fat, because the amount of energy attenuation when the treatment beam B passes through the fat is small, the water equivalent thickness has a small value. For example, when the CT value is a value indicating air, because there is no energy attenuation when the treatment beam B passes through the air, the water equivalent thickness becomes “0.” By converting each CT value included in the CT image into a water equivalent thickness, amounts of energy attenuation based on pixels located on the path of the treatment beam B can be indicated according to the same reference. As a conversion formula for converting the CT value into the water equivalent thickness, for example, a regression equation based on experimentally obtained nonlinear conversion data may be used. Various literature has been published in relation to experimentally obtained nonlinear conversion data.

The difference calculator 140 outputs information including the calculated difference image or a difference (deviation amount) between the first fluoroscopic image and the 2P^{th} fluoroscopic image indicated in the difference image (hereinafter referred to as a “difference image” including the information) to the differential statistical quantity calculator 150.

The differential statistical quantity calculator 150 calculates a statistical quantity (hereinafter referred to as a “differential statistical quantity”) of a difference (deviation amount) between the first fluoroscopic image and the 2P^{th} fluoroscopic image represented by the difference image output by the difference calculator 140. The differential statistical quantity is, for example, a numerical value (data) such as an average of absolute values of pixel values of the difference image, a standard deviation, an intermediate value, or a maximum value. The differential statistical quantity may be, for example, a distribution of pixel values of a difference image or a value (data) for generating a graph such as a histogram. The differential statistical quantity may be, for example, a cumulative distribution of errors, or a value (data) for generating a graph such as a cumulative histogram. A differential statistical quantity is an example of a “statistical quantity.”

A range in which the differential statistical quantity calculator 150 calculates the differential statistical quantity is, for example, the entire region of the difference image. The range in which the differential statistical quantity calculator 150 calculates the differential statistical quantity, for example, may be limited to a range narrowed down to a region such as a tumor of the patient P included in the difference image, other organs, or a designated region such as a PTV determined in the treatment planning phase. The range in which the differential statistical quantity calculator 150 calculates the differential statistical quantity, for example, may be particularly narrowed down to only the boundary portion even within the PTV. When the differential statistical quantity calculator 150 narrows down the range for calculating the differential statistical quantity, the difference calculator 140 may also set the range for calculating the difference image to a range corresponding to the range in which the differential statistical quantity calculator 150 calculates the differential statistical quantity. In this case, an amount of calculation required for the difference calculator 140 to calculate the difference image can be reduced. The range in which the differential statistical quantity calculator 150 calculates the differential statistical quantity, for example, may be changed with the irradiation direction or the irradiation range of the treatment beam B. For example, the differential statistical quantity calculator 150 may calculate the differential statistical quantity by narrowing down a region on the front side of the tumor (a side close to the treatment beam irradiation gate 16) and a region on the back side of the tumor (a side far from the treatment beam irradiation gate 16) in accordance with a distance from the treatment beam irradiation gate 16 to the tumor in the body of the patient P. In other words, the differential statistical quantity calculator 150 may be configured to calculate a differential statistical quantity on the basis of an irradiation direction of the treatment beam B and a spatial positional relationship between the tumor and the treatment beam irradiation gate 16 from which the treatment beam B is radiated. Thereby, the differential statistical quantity calculator 150 can calculate a differential statistical quantity reflecting a condition of an error of a dose of treatment beams B loosely set on the front side of the tumor and strictly set on the back side of the tumor in the treatment planning phase so that the tumor does not remain without radiation to the back side of the tumor or so that the treatment beam B is not radiated to the region of normal tissue on the back side of the tumor. The range in which the differential statistical quantity calculator 150 calculates the differential statistical quantity may be, for example, a division region obtained in segmentation according to image processing performed during radiation treatment in the treatment system 1. For example, the differential statistical quantity calculator 150 may divide anatomical tissue into segment regions to calculate the differential statistical quantity. The differential statistical quantity calculated by the differential statistical quantity calculator 150 may be a vector value obtained by combining the above segment regions.

The differential statistical quantity calculator 150 outputs data indicating the calculated differential statistical quantity (hereinafter simply referred to as a “differential statistical quantity”). The differential statistical quantity output by the differential statistical quantity calculator 150 is presented to the user by, for example, the medical image processing device 100, and is referred to when it is determined whether or not the alignment process of the medical image processing device 100 is being performed correctly. The method of presenting the differential statistical quantity in the medical image processing device 100 to the user in this case may be a method of displaying an image and/or a numerical value indicating a differential statistical quantity on, for example, the display device D or may be a method of displaying an image and/or a numerical value indicating a differential statistical quantity on a liquid crystal display (not shown) provided in the medical image processing device 100 or the like.

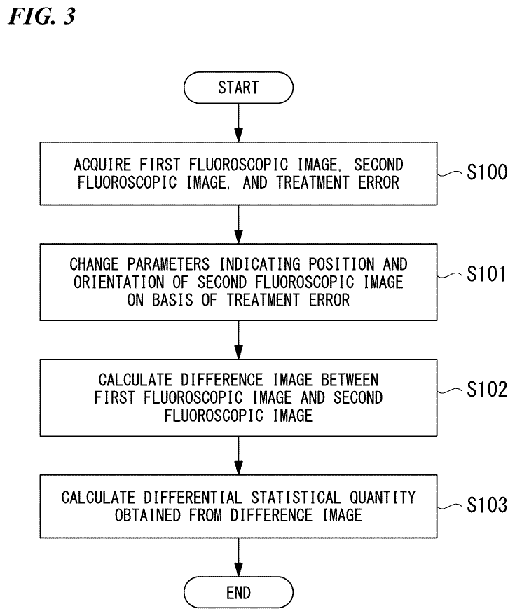

Hereinafter, a flow of a process of outputting a differential statistical quantity (hereinafter referred to as a “differential statistical quantity calculation process”) in the medical image processing device 100 will be described. FIG. 3 is a flowchart showing the flow of the process of outputting the differential statistical quantity in the medical image processing device 100. Before the medical image processing device 100 performs the differential statistical quantity calculation process, i.e., before radiation treatment is performed (for example, about one week prior), a treatment plan is made on the basis of a first fluoroscopic image that has been captured. Furthermore, a second fluoroscopic image is captured immediately before the medical image processing device 100 performs the differential statistical quantity calculation process, i.e., immediately before the radiation treatment starts. In the radiation treatment, the treatment beam B may be radiated multiple times (even if it is not on the same day) to treat the same patient P. For this reason, in the case of second and subsequent radiation treatment for the same patient P, the second fluoroscopic image in which the position of the patient P has been aligned during the previous treatment is used as the first fluoroscopic image and another treatment plan may be made. Also, when the medical image processing device 100 performs the differential statistical quantity calculation process, an alignment process is performed.

Because the medical image processing device 100 mainly focuses on the calculation of a differential statistical quantity to be referred to when a user determines whether or not an alignment process of aligning the position of the patient P has been correctly performed when radiation treatment is performed in the treatment system 1, more detailed description related to a process when respective images (here, CT images), i.e., the first fluoroscopic image and the second fluoroscopic image, are captured or an alignment process will be omitted. In the following description, it is assumed that the treatment plan based on the first fluoroscopic image is completed, the second fluoroscopic image is captured in the treatment system 1, and at least one alignment process has already been completed. Therefore, in the following description, it is assumed that the second fluoroscopic image is a second fluoroscopic image after the alignment process.

First, when the medical image processing device 100 starts a differential statistical quantity calculation process, the first image acquirer 110 acquires the first fluoroscopic image and parameters indicating the position and orientation of the first fluoroscopic image, the second image acquirer 120 acquires a second fluoroscopic image and parameters indicating the position and orientation of the second fluoroscopic image, and the treatment error acquirer 130 acquires a treatment error (step S100). The first image acquirer 110 outputs the acquired first fluoroscopic image and the parameters of the first fluoroscopic image to the difference calculator 140. The second image acquirer 120 outputs the acquired second fluoroscopic image and the parameters of the second fluoroscopic image to the difference calculator 140. The treatment error acquirer 130 outputs the acquired treatment error to the difference calculator 140.

Next, the difference calculator 140 changes the parameters indicating the position and orientation of the second fluoroscopic image after the alignment process on the basis of the treatment error from the treatment error acquirer 130 (step S101). That is, the difference calculator 140 applies a virtual perturbation to the body posture of the patient P shown in the second fluoroscopic image after the alignment process.

Next, the difference calculator 140 calculates a difference image having a difference between the first fluoroscopic image and the second fluoroscopic image (a 2P^{th} fluoroscopic image) in which a virtual perturbation is applied to the body posture of the patient P (step S102). The difference calculator 140 outputs the calculated difference image to the differential statistical quantity calculator 150.

Next, the differential statistical quantity calculator 150 calculates the differential statistical quantity obtained from the difference image output by the difference calculator 140 (step S103). The differential statistical quantity calculator 150 outputs the calculated differential statistical quantity. Also, the medical image processing device 100 presents the differential statistical quantity output by the differential statistical quantity calculator 150 to the user.

According to this process, in the differential statistical quantity calculation process of the medical image processing device 100, the difference calculator 140 calculates a difference image by changing the parameters indicating the position and orientation of the second fluoroscopic image after the alignment process on the basis of the treatment error (by applying the virtual perturbation) and the differential statistical quantity calculator 150 calculates the differential statistical quantity on the basis of the difference image. The medical image processing device 100 iterates the above-described differential statistical quantity calculation process (or performs differential statistical quantity calculation processes equal in number to the number of times a virtual perturbation is applied) to calculate a plurality of difference images and calculates a differential statistical quantity based on each difference image. Also, the medical image processing device 100 presents each calculated differential statistical quantity to the user. Thereby, the user can refer to the presented differential statistical quantity to determine whether or not the current alignment process of the medical image processing device 100 has been performed correctly. If it is determined that this alignment process of the medical image processing device 100 has not been performed correctly, the user can instruct the medical image processing device 100 to perform the alignment process again.

Here, the differential statistical quantity presented by the medical image processing device 100, for example, is presented in addition to or instead of an image for visually confirming whether or not a target site of the radiation treatment matches the position at the time of the treatment plan by superimposing the first fluoroscopic image and the second fluoroscopic image after the alignment process, which are presented in a medical image processing device provided in a conventional treatment system, in a semi-transparent manner. Moreover, the differential statistical quantity presented by the medical image processing device 100 is data obtained by quantitatively expressing a deviation (error) between the first fluoroscopic image and the second fluoroscopic image after the alignment process, in other words, a degree of matching of the position of the patient P or the like. Thus, although the position of the patient P after the alignment process is ideally aligned even in a conventional treatment system, it is difficult for the user to make the determination from an overlapping state of the two fluoroscopic images, i.e., the first fluoroscopic image and the second fluoroscopic image after the alignment process and it is possible to more easily and accurately determine whether the alignment process of the medical image processing device 100 is being performed correctly. Thereby, for example, as in a conventional treatment system in which the user visually confirms whether or not the alignment process of the medical image processing device 100 is being performed correctly, in the treatment system 1 including the medical image processing device 100, radiation treatment can be performed without any difference in effectiveness depending on the ability of the user conducting the confirmation.

Furthermore, the calculation of the differential statistical quantity presented by the medical image processing device 100 takes into account a treatment error in radiation treatment not appearing in the treatment planning phase and it is possible to determine whether or not the current radiation treatment can be continued even if the body posture of the patient P is shifted during the radiation treatment. Thus, the user can determine whether or not a result of the alignment process of the medical image processing device 100 can be handled, for example, even if there is a change in the patient's state (such as a change in an orientation thereof) that is assumed (estimated) to change over time as the radiation treatment progresses, a movement error of the patient table 12, or the like.

As described above, in the medical image processing device 100, the first image acquirer 110 acquires a first fluoroscopic image of the patient P photographed before treatment and parameters indicating the position and orientation when the first fluoroscopic image has been captured and the second image acquirer 120 acquires a second fluoroscopic image of the patient P photographed immediately before treatment starts and parameters indicating the position and orientation when the second fluoroscopic image has been captured. Furthermore, in the medical image processing device 100, the treatment error acquirer 130 acquires a treatment error in radiation treatment not appearing in the treatment planning phase. Also, in the medical image processing device 100, the difference calculator 140 changes the parameters indicating the position and orientation of the second fluoroscopic image after the alignment process on the basis of the treatment error (by applying a virtual perturbation) and calculates a difference image. Thereafter, in the medical image processing device 100, the differential statistical quantity calculator 150 calculates a differential statistical quantity on the basis of the difference image. Also, the medical image processing device 100 presents the calculated differential statistical quantity to the user. Thereby, in the treatment system 1 including the medical image processing device 100, the user can determine whether or not the current alignment process of the medical image processing device 100 is being performed correctly with reference to the presented differential statistical quantity.

As described above, the medical image processing device 100 includes the first image acquirer 110 configured to acquire a first fluoroscopic image obtained by photographing the inside of the body of the patient P, the second image acquirer 120 configured to acquire a second fluoroscopic image of the inside of the body of the patient P photographed at a timing different from that of the first fluoroscopic image, the treatment error acquirer 130 configured to acquire a treatment error occurring when an alignment process of aligning the position of the patient P shown in the second fluoroscopic image with the position of the patient P shown in the first fluoroscopic image is performed on the basis of the first fluoroscopic image and the second fluoroscopic image or a treatment error occurring in radiation treatment, the difference calculator 140 configured to apply a virtual perturbation to the position of the patient P shown in the second fluoroscopic image on the basis of the treatment error and calculate a difference image between the second fluoroscopic image to which the perturbation is applied and the first fluoroscopic image, and the differential statistical quantity calculator 150 configured to calculate a differential statistical quantity that is a difference between the first fluoroscopic image and the second fluoroscopic image to which the perturbation is applied on the basis of the difference image. Thereby, the medical image processing device 100 can present the differential statistical quantity calculated by the differential statistical quantity calculator 150 to the user.

As described above, the difference calculator 140 may apply perturbations of a plurality of phases to the position of the patient P shown in the second fluoroscopic image and calculate a difference image for each perturbation that has been applied and the differential statistical quantity calculator 150 may calculate a differential statistical quantity corresponding to each of the perturbations on the basis of each difference image. Thereby, the medical image processing device 100 can present each differential statistical quantity calculated by applying the virtual perturbations in the plurality of phases to the user.

As described above, the treatment system 1 includes the medical image processing device 100 and the treatment device 10 having the treatment beam irradiation gate 16 configured to irradiate the patient P with the treatment beam B, the CT photography device 14 configured to capture a first fluoroscopic image and a second fluoroscopic image, and the patient table 12 on which the patient P is placed and fixed. Thereby, the treatment system 1 can perform radiation treatment for the user at the position of the patient P determined by the user with reference to the differential statistical quantity presented by the medical image processing device 100.

### Second Embodiment

Hereinafter, a second embodiment will be described. In the first embodiment, a configuration in which the differential statistical quantity calculated by the differential statistical quantity calculator 150 is presented to the user has been described. That is, a configuration in which the user determines whether or not the alignment process of the medical image processing device 100 is being performed correctly has been described. In the second embodiment, a configuration and method in which it is possible to automatically determine whether or not it is necessary to adjust the position of the patient P after the alignment process as an aid to the user's determination will be described.

The configuration of the treatment system including the medical image processing device of the second embodiment is a configuration in which the medical image processing device 100 is replaced with the medical image processing device 200 of the second embodiment in the configuration of the treatment system 1 including the medical image processing device 100 of the first embodiment shown in FIG. 1. In the following description, the treatment system including the medical image processing device 200 will be referred to as a “treatment system 2.”

In the following description, constituent elements identical to those of the components of the treatment system 1 including the medical image processing device 100 are denoted by the same reference signs in the constituent elements of the treatment system 2 including the medical image processing device 200 and a redundant detailed description thereof will be omitted.

Like the medical image processing device 100, the medical image processing device 200 calculates a differential statistical quantity in the differential statistical quantity calculation process and presents the differential statistical quantity to a user. Furthermore, the medical image processing device 200 determines whether or not it is necessary to adjust the position of a patient P on the basis of the calculated differential statistical quantity and presents a determination result to the user.

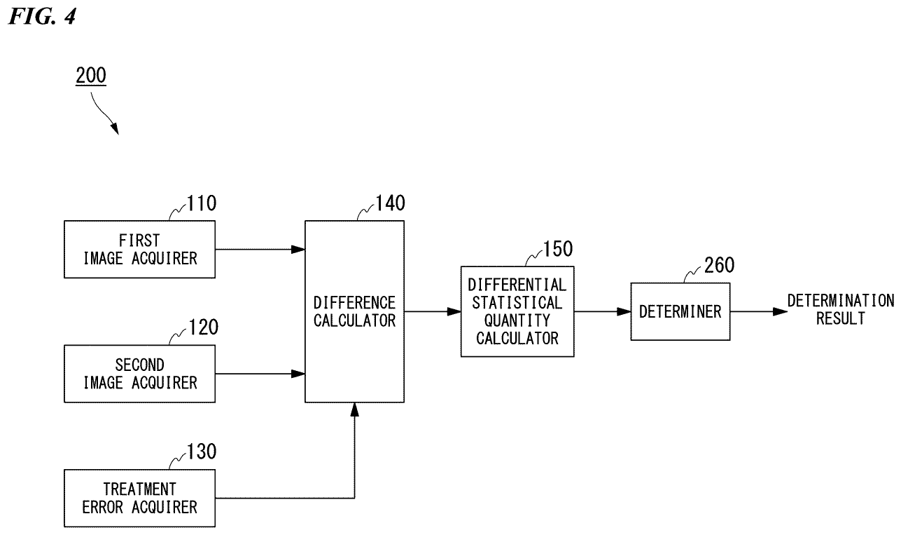

Hereinafter, a configuration of the medical image processing device 200 constituting the treatment system 2 will be described. FIG. 4 is a block diagram showing a schematic configuration of the medical image processing device 200 of the second embodiment. The medical image processing device 200 includes, for example, a first image acquirer 110, a second image acquirer 120, a treatment error acquirer 130, a difference calculator 140, a differential statistical quantity calculator 150, and a determiner 260. The medical image processing device 200 has a configuration in which the determiner 260 is added to the medical image processing device 100.

On the basis of the differential statistical quantity output by the differential statistical quantity calculator 150, the determiner 260 determines whether or not it is necessary to adjust the position of the patient P in the alignment process of the medical image processing device 200. The determination of whether or not it is necessary to adjust the position of the patient P in the determiner 260 is made, for example, by comparing the differential statistical quantity with a predetermined threshold value in a magnitude relationship. The determination of whether or not it is necessary to adjust the position of the patient P in the determiner 260, for example, may be made by comparing an output value of a model expression such as a weighted sum of a plurality of differential statistical quantities with the predetermined threshold value in a magnitude relationship. The determination of whether or not it is necessary to adjust the position of the patient P in the determiner 260, for example, may be made by calculating a ratio of pixel values greater than or equal to a predetermined difference value with respect to the distribution of pixel values in the difference image and comparing the calculated value with the predetermined threshold value in the magnitude relationship. For example, the predetermined threshold value is input to the medical image processing device 200 or the determiner 260 by the user manipulating the user interface (not shown).

The determiner 260 outputs a result of determining whether or not it is necessary to adjust the position of the patient P (a determination result). The determination result output by the determiner 260 is presented to, for example, the user, by the medical image processing device 200, and is referred to when the user determines whether to perform the alignment process again. A method of presenting the determination result of the medical image processing device 200 to the user in this case may be, for example, a method of displaying an image indicating the determination result on a display device D, or a liquid crystal display (not shown) provided in the medical image processing device 200, a method of expressing a determination result by turning on/off an LED or lamp provided in the medical image processing device 200, changing color, or the like.

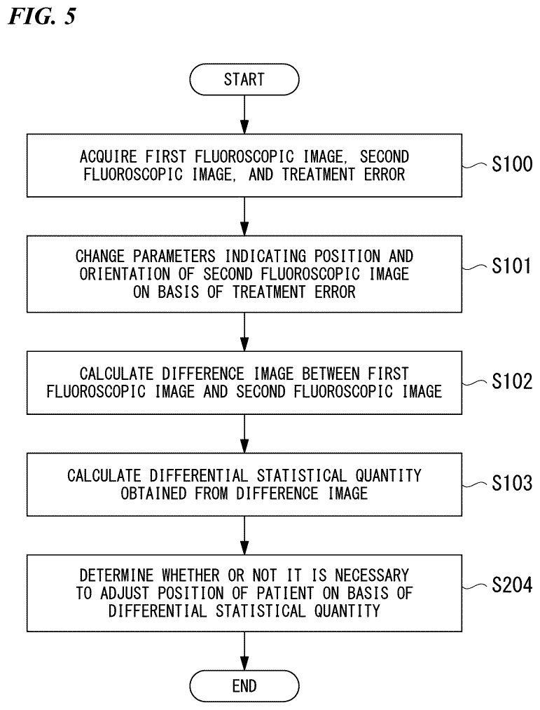

Hereinafter, in the medical image processing device 200, a flow of a process of determining whether it is necessary to adjust the position of the patient P (hereinafter referred to as an “adjustment determination process”) will be described. FIG. 5 is a flowchart showing the flow of the process of determining whether it is necessary to adjust the position of the patient P in the medical image processing device 200. In the adjustment determination process of the medical image processing device 200, because the process until the differential statistical quantity calculator 150 calculates the differential statistical quantity is similar to the differential statistical quantity calculation process in the medical image processing device 100, a redundant detailed description thereof will be omitted.

When the differential statistical quantity calculator 150 calculates the differential statistical quantity in step S103, the differential statistical quantity calculator 150 outputs the calculated differential statistical quantity to the determiner 260. The determiner 260 determines whether or not it is necessary to adjust the position of the patient P on the basis of the differential statistical quantity output by the differential statistical quantity calculator 150 (step S204). The determiner 260 outputs a determination result. Also, the medical image processing device 200 presents the determination result output by the determiner 260 to the user.

According to such configuration, operation, and process, like the medical image processing device 100, the medical image processing device 200 determines whether or not it is necessary to adjust the position of the patient P on the basis of the differential statistical quantity calculated by the differential statistical quantity calculator 150 in the adjustment determination process. Also, the medical image processing device 200 presents the calculated differential statistical quantity and a determination result obtained by determining whether or not it is necessary to adjust the position of the patient P to the user. Thereby, in the medical image processing device 200, as in the medical image processing device 100, the user can also quantitatively determine the degree of matching of the position of the patient P and the like and can use the support for determining whether or not it is necessary to adjust the position of the patient P after the alignment process. If it is determined that the current alignment process of the medical image processing device 200 is not being performed correctly or if it is determined that it is necessary to adjust the position of the patient P, the user can instruct the medical image processing device 200 to perform the alignment process again or to adjust the position of the patient P. Thereby, the treatment system 2 including the medical image processing device 200 can perform more effective radiation treatment like the treatment system 1 including the medical image processing device 100.

As described above, even in the medical image processing device 200, as in the medical image processing device 100, the first image acquirer 110 acquires a first fluoroscopic image of the patient P photographed before treatment and parameters indicating the position and orientation when the first fluoroscopic image is captured and the second image acquirer 120 acquires a second fluoroscopic image of the patient P photographed immediately before treatment starts and parameters indicating the position and orientation when the second fluoroscopic image is captured. Furthermore, in the medical image processing device 200, as in the medical image processing device 100, the treatment error acquirer 130 acquires a treatment error in radiation treatment not appearing in the treatment planning phase. Also, in the medical image processing device 200, as in the medical image processing device 100, the difference calculator 140 changes the parameters indicating the position and orientation of the second fluoroscopic image after the alignment process on the basis of the treatment error (by applying a virtual perturbation) and calculates a difference image. Subsequently, in the medical image processing device 200, as in the medical image processing device 100, the differential statistical quantity calculator 150 calculates the differential statistical quantity on the basis of the difference image. Also, in the medical image processing device 200, as in the medical image processing device 100, the calculated differential statistical quantity is presented to the user. Furthermore, in the medical image processing device 200, the determiner 260 determines whether or not it is necessary to adjust the position of the patient P on the basis of the differential statistical quantity calculated by the differential statistical quantity calculator 150. Thereby, in the treatment system 2 including the medical image processing device 200, as in the treatment system 1 including the medical image processing device 100, the user can determine whether or not the current alignment process of the medical image processing device 200 is being performed correctly with reference to the presented differential statistical quantity. Furthermore, in the treatment system 2 including the medical image processing device 200, the user can determine whether or not to adjust the position of the patient P with reference to the determination result of the determiner 260.

Moreover, in the medical image processing device 200, because the determiner 260 quantitatively automatically determines whether or not it is necessary to adjust the position of the patient P, it is possible to implement a treatment system that automatically starts adjusting the position of the patient P at a higher speed than when the user performs the determination and the instruction. In other words, it is possible to implement a treatment system that automatically performs the alignment process of aligning the patient P at a position suitable for radiation treatment.

As described above, the medical image processing device 200 further includes the determiner 260 configured to determine a result of the alignment process on the basis of the differential statistical quantity with respect to the medical image processing device 100 of the first embodiment. Thereby, the medical image processing device 200 can present a determination result of determining whether or not it is necessary to adjust the position of the patient P in the determiner 260 to the user.

### Third Embodiment

Hereinafter, a third embodiment will be described. In the first embodiment and the second embodiment, the configuration, operation, and process of the medical image processing device 100 or the medical image processing device 200 have been described under the assumption that at least one alignment process has already been completed. In the third embodiment, a medical image processing device including a configuration in which an alignment process is performed will be described.

The configuration of the treatment system including the medical image processing device of the third embodiment is a configuration in which the medical image processing device 100 is replaced with a medical image processing device 300 of the third embodiment in the configuration of the treatment system 1 including the medical image processing device 100 of the first embodiment shown in FIG. 1. In the following description, the treatment system including the medical image processing device 300 is referred to as a “treatment system 3.”

In the following description, constituent elements identical to those of the treatment system 1 including the medical image processing device 100 or the treatment system 2 including the medical image processing device 200 of the second embodiment are denoted by the same reference signs in the constitute elements of the treatment system 3 including the medical image processing device 300 and redundant detailed description thereof will be omitted.

Like the medical image processing device 100 or the medical image processing device 200, the medical image processing device 300 performs an alignment process for aligning the position of the patient P when radiation treatment is performed on the basis of a CT image output by a CT photography device 14 and outputs a movement control signal for moving a patient table 12 so that an irradiation direction of a treatment beam B radiated from a treatment beam irradiation gate 16 is aligned with a direction set in the treatment planning phase. Also, like the medical image processing device 100 or the medical image processing device 200, the medical image processing device 300 calculates a differential statistical quantity in a differential statistical quantity calculation process and presents the differential statistical quantity to the user. Furthermore, like the medical image processing device 200, the medical image processing device 300 determines whether or not it is necessary to adjust the position of the patient P on the basis of the calculated differential statistical quantity and presents a determination result to the user.

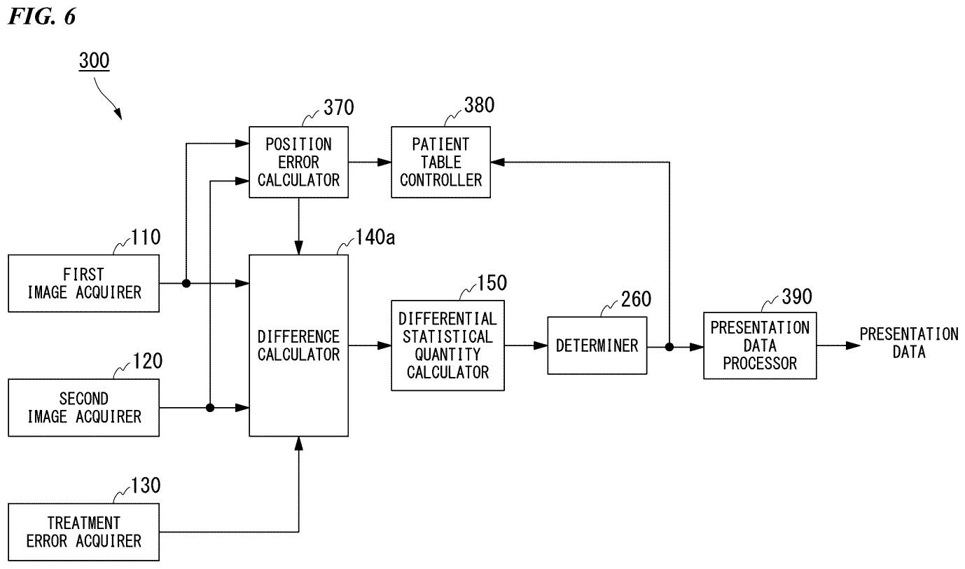

Hereinafter, a configuration of the medical image processing device 300 constituting the treatment system 3 will be described. FIG. 6 is a block diagram showing a schematic configuration of the medical image processing device 300 of the third embodiment. The medical image processing device 300 includes a first image acquirer 110, a second image acquirer 120, a treatment error acquirer 130, a difference calculator 140a, a differential statistical quantity calculator 150, a determiner 260, a position error calculator 370, a patient table controller 380, and a presentation data processor 390. The medical image processing device 300 has a configuration in which the position error calculator 370, the patient table controller 380, and the presentation data processor 390 are added to the medical image processing device 200. Accordingly, the difference calculator 140 provided in the medical image processing device 200 is replaced with the difference calculator 140a.

The position error calculator 370 performs an alignment process on the basis of a first fluoroscopic image and parameters output by the first image acquirer 110 and a second fluoroscopic image and parameters output by the second image acquirer 120. More specifically, the position error calculator 370 acquires the first fluoroscopic image and the parameters indicating the position and orientation of the first fluoroscopic image and acquires the second fluoroscopic image and the parameters indicating the position and orientation of the second fluoroscopic image. Also, the position error calculator 370 calculates the amount of movement related to the position and orientation of the second fluoroscopic image so that the position of the patient when the second fluoroscopic image is captured is aligned with the position of the patient when the acquired first fluoroscopic image is captured.

Here, adjusting the position of the patient when the second fluoroscopic image is captured to the position of the patient when the first fluoroscopic image is captured is calculating similarity between the first fluoroscopic image and the second fluoroscopic image while changing the parameters indicating the position and orientation of the second fluoroscopic image in various ways and solving a problem for obtaining a parameter for increasing the similarity to a highest level. For this reason, in the alignment process of the position error calculator 370, the efficiency of the selection of similarity and the search for parameters significantly affects the accuracy of the alignment of the patient P and the calculation time (processing time). The similarity is, for example, a scalar value obtained by calculating a difference image while changing the parameters of the second fluoroscopic image as in the difference calculator 140 and obtaining a difference between the first fluoroscopic image and the second fluoroscopic image obtained by changing the parameter (an amount of deviation therebetween) indicated in the calculated difference image. As a parameter search method, optimization methods such as a gradient method, a Newtonian method, and a Lucas-Kanade method (LK method) are used.

The position error calculator 370 outputs a calculated amount of movement related to the position and orientation of the second fluoroscopic image as a result of the alignment process to the difference calculator 140a and the patient table controller 380.

Thereby, like the difference calculator 140, the difference calculator 140a moves the parameters of the second fluoroscopic image on the basis of the amount of movement indicated in a processing result output by the position error calculator 370 and calculates a difference image by applying a virtual perturbation using the second fluoroscopic image as the second fluoroscopic image after the alignment process.

The patient table controller 380 generates a movement control signal for controlling a translation mechanism and a rotation mechanism provided in the patient table 12 on the basis of an amount of movement indicated in a processing result output by the position error calculator 370 and a result of determining whether or not it is necessary to adjust the position of the patient P output by the determiner 260. The patient table controller 380 outputs the generated movement control signal to the treatment device 10. Thereby, the treatment device 10 controls the translation mechanism and the rotation mechanism in accordance with the movement control signal output by the patient table controller 380 and moves the patient table 12 so that the current body posture of the patient P fixed to the patient table 12 is close to the body posture of the patient P in the treatment planning phase. Thereby, in the treatment system 3 including the medical image processing device 300, the irradiation direction of the treatment beam B radiated from the treatment beam irradiation gate 16 to the patient P is adjusted to a direction set in the treatment planning phase and radiation treatment can be performed.

The presentation data processor 390 generates presentation data for presenting a calculation result or information of the medical image processing device 300 to the user. The presentation data is, for example, the first fluoroscopic image and parameters output by the first image acquirer 110, the second fluoroscopic image and parameters output by the second image acquirer 120, the treatment error output by the treatment error acquirer 130, the difference image output by the difference calculator 140 (including information indicating the difference between the first fluoroscopic image and the 2P^{th} fluoroscopic image (the amount of deviation therebetween) indicated in the difference image), the differential statistical quantity output by the differential statistical quantity calculator 150, and the determination result output by the determiner 260. The presentation data processor 390 presents information to the user by generating an image indicating the information as presentation data and displaying the presentation data on the display device D.

Here, an example of the presentation data generated by the presentation data processor 390 and displayed on the display device D will be described. As described above, the presentation data processor 390 causes information such as the first fluoroscopic image, the second fluoroscopic image, the treatment error, the difference image, the differential statistical quantity, and the determination result to be displayed on the display device D. Hereinafter, an example in which the presentation data processor 390 presents a differential statistical quantity as a representative of these information items to the user will be described.

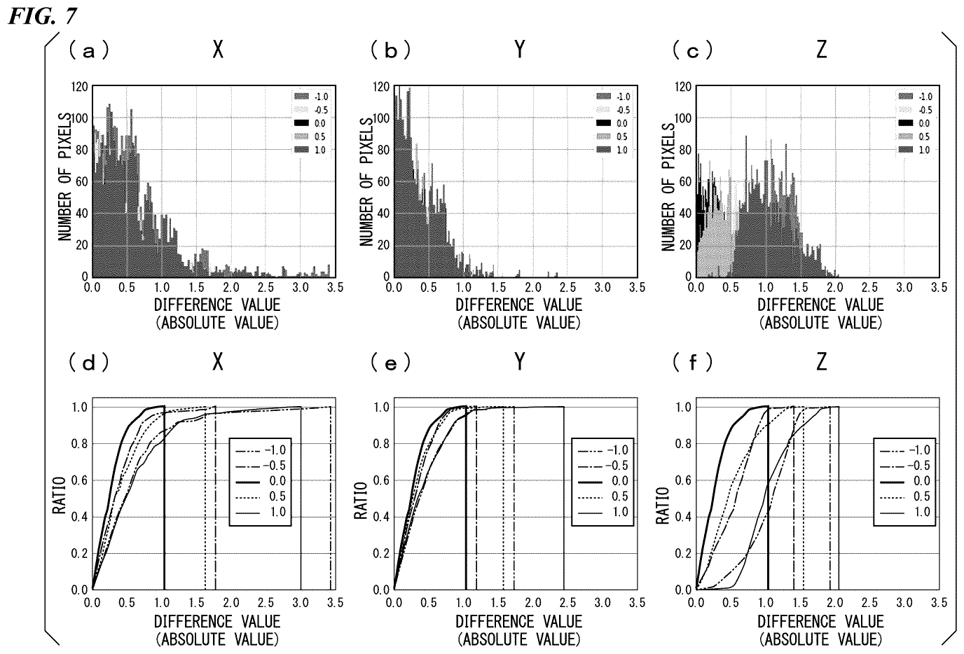

FIG. 7 is a diagram showing an example of presentation data generated by the presentation data processor 390 provided in the medical image processing device 300. In FIG. 7, an example of presentation data in which the differential statistical quantity calculated by the differential statistical quantity calculator 150 is represented graphically is shown. In FIG. 7, an example in which the movement error (treatment error) of the patient table 12 is exaggerated is shown for description. More specifically, in (a) to (c) of FIG. 7, an example in which the difference calculator 140a calculates a difference image by applying treatment errors of ±0.5 mm and ±1.0 mm (a movement error of the patient table 12) as a virtual perturbation to each of the three axial directions of the translational mechanism and the differential statistical quantity calculator 150 uses a differential statistical quantity obtained by calculating an error of a water equivalent thickness (water equivalent path length (WEL)) in each pixel of the difference image as a histogram indicating an error distribution is shown. In (d) to (f) of FIG. 7, an example in which differential statistical quantities identical to those of (a) to (c) in FIG. 7 are used as cumulative histograms indicating the cumulative distribution of errors is shown. For example, (a) and (d) of FIG. 7 provide virtual perturbations in an X-axis direction (see FIG. 1), (b) and (e) of FIG. 7 provide virtual perturbations in a Y-axis direction (see FIG. 1), and (c) and (f) of FIG. 7 provide virtual perturbations in a Z-axis direction (see FIG. 1). In each of (a) to (c) of FIG. 7, the horizontal axis represents a difference value (absolute value) of the water equivalent thickness and the vertical axis represents the number of pixels of the same difference value. In each of (d) to (f) of FIG. 7, the horizontal axis represents a difference value (absolute value) of the water equivalent thickness and the vertical axis represents a proportion of pixels with the same difference value. In (a) to (f) of FIG. 7, the differential statistical quantity when the alignment process is completed, i.e., when the perturbation is 0.0 mm, is also shown.

For example, from the cumulative histograms shown in (d) to (f) of FIG. 7, it can be seen that the difference value of the water equivalent thickness is within about 1.0 mm in all three axial directions of the translational mechanism and about 90% thereof is within about 0.5 mm in a state in which the alignment process is completed. Also, from the histograms shown in (a) to (c) of FIG. 7 and the cumulative histograms shown in (d) to (f) of FIG. 7, it can be seen that the difference value of the water equivalent thickness increases when a treatment error occurs in the direction of any axis of the translational mechanism. More specifically, it can be seen that the difference value of the water equivalent thickness when a treatment error of the translational mechanism occurs in the X-axis direction is larger than that when a treatment error thereof occurs in the Y-axis direction or the Z-axis direction. On the other hand, when a treatment error of the translational mechanism occurs in the Z-axis direction, it can be seen that a change in the difference value of the water equivalent thickness due to the treatment error is small.

Although an example of a graph corresponding to each of the three axis directions (the X-axis direction, the Y-axis direction, and the Z-axis direction) of the translational mechanism is shown in (a) to (f) of FIG. 7, the treatment device 10 can move the patient table 12 with six degrees of freedom using the translational mechanism and the rotation mechanism. That is, the treatment device 10 can be moved at each rotational angle (yaw, roll, and pitch) around the three axes of the rotation mechanism in addition to the three axis directions of the translation mechanism. Therefore, the presentation data processor 390 may generate a graph corresponding to each rotational angle around the three axes of the rotation mechanism as in (a) to (f) of FIG. 7. In each graph shown in (a) to (f) of FIG. 7, the differential statistical quantity in the state in which the alignment process is completed (the perturbation is 0.0 mm) and the differential statistical quantity when a virtual perturbation is applied are shown together. That is, in each graph shown in (a) to (f) of FIG. 7, a plurality of differential statistical quantities are shown at the same time. However, each of the graphs shown in (a) to (f) of FIG. 7 may indicate any one differential statistical quantity.

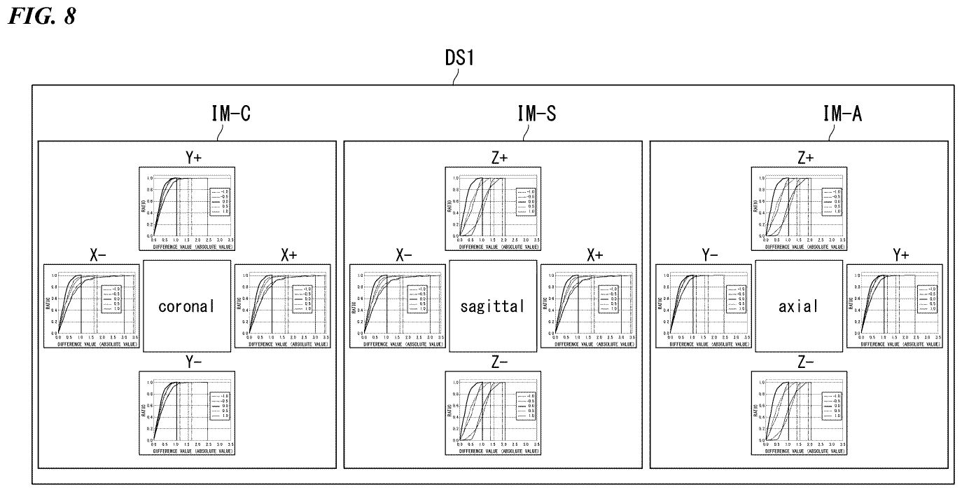

The presentation data processor 390 generates an image (display image) indicating this information and causes the display device D to display the generated image. FIG. 8 is a diagram showing an example of a display screen in which the presentation data processor 390 provided in the medical image processing device 300 causes the display device D to display the presentation data. In FIG. 8, an example in which the presentation data processor 390 causes a display screen DS1 of the display device D to display an image of the presentation data is shown.

“Coronal,” “sagittal,” and “axial” shown in FIG. 8 represent cross-sectional images of the patient P. More specifically, “coronal” represents a coronal cross-section (frontal cross-section) for dividing the patient P into two parts, i.e., a front side (thoracic side) and a back side (dorsal side) in the left/right direction, in other words, a cross-section (an X-Y cross-section in the room coordinate system shown in FIG. 1) for seeing the patient P from right front or right back. “Sagittal” represents a sagittal cross-section for dividing the patient P into two parts, i.e., left and right parts, in the anteroposterior direction, in other words, a cross-section of the patient P viewed in a directly horizontal direction (an X-Z cross-section in the room coordinate system shown in FIG. 1). “Axial” represents a horizontal cross-section (axial cross-section) for dividing the patient P into two parts, i.e., an upper side (cephalic side) and a lower side (foot side), in the left/right direction, in other words, a cross-section viewed so that the patient P is looked down from the head side or the patient P is looked up from the foot side (a Y-Z cross-section in the room coordinate system shown in FIG. 1). The presentation data processor 390 may arrange the corresponding cross-sectional image (which may be a difference image of the corresponding cross-section) in a central region where “coronal,” “sagittal,” or “axial” is shown in a coronal cross-sectional image IM-C, a sagittal cross-sectional image IM-S, and a horizontal cross-sectional image IM-A arranged on the display screen DS1.

In the coronal cross-sectional image IM-C, the sagittal cross-sectional image IM-S, and the horizontal cross-sectional image IM-A, cumulative histograms arranged as “X+,” “X−,” “Y+,” “Y−,” “Z+,” and “Z−” are, for example, cumulative histograms similar to the cumulative histogram of the differential statistical quantity shown in FIG. 7. However, the cumulative histograms arranged in the coronal cross-sectional image IM-C, the sagittal cross-sectional image IM-S, and the horizontal cross-sectional image IM-A are different from the cumulative histograms shown in FIG. 7 because virtual perturbations applied in the three axis directions of the translation mechanism are divided in a positive (+) direction and a negative (−) direction, the difference value of the water equivalent thickness of the horizontal axis is not an absolute value, but is a value of the corresponding direction. That is, each of the cumulative histograms arranged in the coronal cross-sectional image IM-C, the sagittal cross-sectional image IM-S, and the horizontal cross-sectional image IM-A is a cumulative histogram of differential statistical quantities obtained by calculating a difference image in which a treatment error (a movement error of the patient table 12) as a virtual perturbation is separately given in a positive (+) direction and a negative (−) direction with respect to each of the three axes of the translation mechanism in the difference calculator 140a and calculating an error in the water equivalent thickness at each pixel of the difference image in the differential statistical quantity calculator 150.

As shown in FIG. 8, the presentation data processor 390 generates an image obtained by arranging a cross-sectional image IM in which each cross-sectional image and a cumulative histogram corresponding to each direction (axial direction) of the cross-sectional image are shown in a positional relationship that is easy for the user to intuitively confirm and causes the display device D to display the generated image. Thereby, from each image IM arranged on the display screen DS1, the user can quantitatively confirm an amount of change in the error of the water equivalent thickness when a shift occurs due to a movement error at the actual position of the patient table 12 for which the alignment process has been completed. Also, the user can decide whether or not to start radiation treatment on the basis of an amount of change that has been confirmed. More specifically, for example, the user can start radiation treatment at the current position of the patient table 12 when the maximum value of the difference value of the water equivalent thickness in each direction shown in the coronal cross-sectional image IM-C, the sagittal cross-sectional image IM-S, and the horizontal cross-sectional image IM-A is within the allowable range and determine a process of finely adjusting the position of the patient table 12 and then starting radiation treatment and the like when it is outside of the allowable range. Fine adjustment of the position of the patient table 12 is performed, for example, by the user manipulating the user interface (not shown).

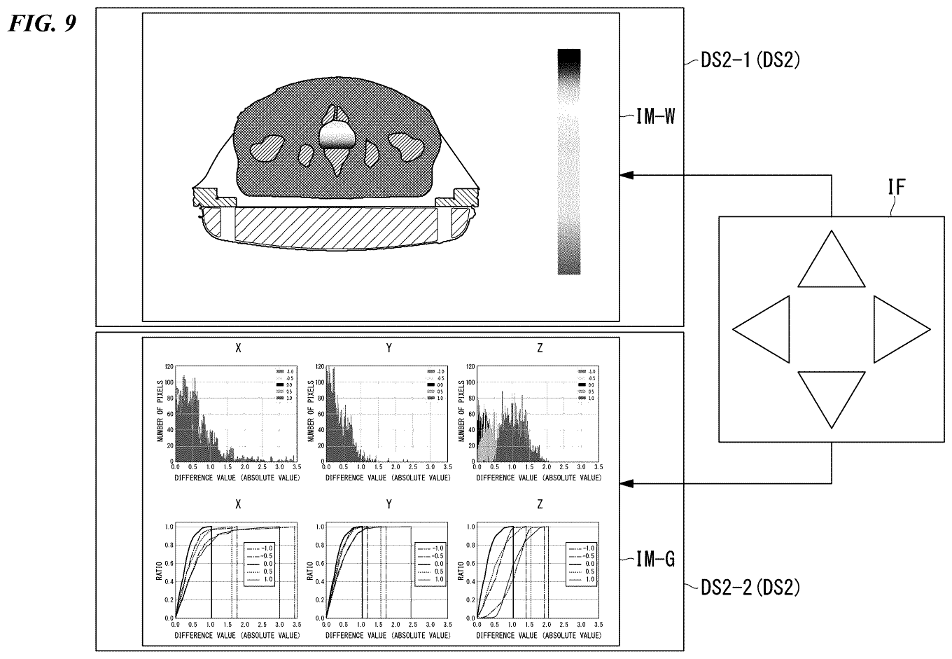

FIG. 9 is a diagram showing an example of another display screen in which the presentation data processor 390 provided in the medical image processing device 300 causes the display device D to display the presentation data. In FIG. 9, for example, an example in which the display screen DS2 is switched in accordance with a manipulation on the user interface IF by the user is shown. More specifically, an example in which the screen is switched between a display screen DS2-1 and a display screen DS2-2 in accordance with a manipulation on the user interface IF (the cross key in FIG. 9) by the user is shown.

The display screen DS2-1 is a display screen DS2 obtained by arranging a cross-sectional image IM-W, which is a difference image between a first fluoroscopic image and a second fluoroscopic image when the CT image is converted into a water equivalent thickness and the water equivalent thickness to a region near a tumor (PTV) is calculated in a line integration process in which the irradiation direction (here, the upward direction) of the treatment beam B is taken into account. The presentation data processor 390 may superimpose information of the treatment planning phase, such as the outline line of a tumor (GTV), on the cross-sectional image IM-W. The display screen DS2-2 is a display screen DS2 obtained by arranging a graph image IM-G representing a graph of an error distribution which is one of differential statistical quantities calculated with respect to the cross-sectional image IM-W by the differential statistical quantity calculator 150 (here, a graph similar to the histogram and the cumulative histogram shown in FIG. 7). For example, the presentation data processor 390 switches the display screen DS2 to be displayed on the display device D to the display screen DS2-1 when the user presses a manipulation button on the upper side of the user interface IF and switches the display screen DS2 to be displayed on the display device D to the display screen DS2-2 when the user presses a manipulation button on the lower side of the user interface IF. Thereby, the user can quantitatively confirm the current alignment state of the patient P and an amount of change in the error of the water equivalent thickness when a deviation caused by the movement error occurs from the state from the images IM arranged on the display screen DS2. Also, the user can determine whether or not to start radiation treatment on the basis of the confirmed state.

Although an example of the display screen DS when the presentation data processor 390 presents information to the user is shown as shown in FIGS. 8 and 9, the display screen DS for presenting information to the user is not limited to the display screen DS as shown in FIGS. 8 and 9. That is, the presentation data processor 390 can generate various display screens DS on the basis of information to be provided to the user and cause the display device D to display the display screens DS.

For example, the presentation data processor 390 may change the graph to be arranged in the graph image IM-G when the user presses the manipulation button on the right or left side of the user interface IF in a state in which the display screen DS2-2 as shown in FIG. 9 is displayed on the display device D. More specifically, the presentation data processor 390 may be configured to change the graphs of the three axis directions (the X-axis direction, the Y-axis direction, and the Z-axis direction) of the translation mechanism arranged in the current graph image IM-G to the graphs of the rotational angles (yaw, roll, and pitch) around the three axes of the rotation mechanism (switch all graphs or sequentially scroll the graphs one by one), for example, when the manipulation button on the right side or the left side of the user interface IF has been pressed.

For example, the presentation data processor 390 may be configured to enlarge the position of the tumor in the cross-sectional image IM-W when any manipulation button of the user interface IF is pressed by the user in a state in which the display screen DS2-1 as shown in FIG. 9 is displayed on the display device D. For example, the presentation data processor 390 may be configured to further display a cursor (not shown) on the enlarged cross-sectional image IM-W and superimpose a pop-up image showing a numerical value (data) such as an average of pixel values of pixels corresponding to the position of the cursor moved by the user with the manipulation button, a standard deviation, an intermediate value, and a maximum value on the current display. For example, the presentation data processor 390 may be configured to display a cursor (not shown) in a state in which the display screen DS2-2 as shown in FIG. 9 is displayed on the display device D and superimpose a pop-up image showing a numerical value (data) such as an average of the absolute values of the pixel values of a difference image corresponding to the position of the cursor on a graph moved by the user using the manipulation button, the standard deviation, the intermediate value, or the maximum value on the current display.

For example, when graphs corresponding to the three axes are displayed as shown on the display screen DS2-2 as shown in FIG. 9, the presentation data processor 390 may be configured to enable the user to select the graph to be displayed or cause graphs corresponding to three axes to be displayed from a graph having a largest error. For example, in the graph image IM-G arranged on the display screen DS2-2 as shown in FIG. 9, the presentation data processor 390 may be configured to cause graphs of rotational angles around the three axes of the rotation mechanism to be displayed at the same time instead of the graph (histogram) of the upper side. That is, for example, the presentation data processor 390 may be configured to simultaneously display cumulative histograms corresponding to all axes of the six degrees of freedom with which the patient table 12 can be moved.

For example, the presentation data processor 390 may be configured to display numerical values (data) such as an average of absolute values of pixel values of a difference image, a standard deviation, an intermediate value, and a maximum value in addition to or instead the graphs (histograms and cumulative histograms) shown in FIGS. 8 and 9.

According to such configuration, operation, and process, in the medical image processing device 300, the position error calculator 370 performs an alignment process and the patient table controller 380 generates a movement control signal to cause the patient table 12 to move to the treatment device 10. Furthermore, in the medical image processing device 300, the presentation data processor 390 generates presentation data for presentation to the user (for example, the display screen DS as shown in FIGS. 8 and 9) and causes the display device D to display the presentation data. Thereby, in the medical image processing device 300, as in the medical image processing device 100 or the medical image processing device 200, the user can also quantitatively determine the degree of matching of the position of the patient P and the like and can use the support for determining whether or not it is necessary to adjust the position of the patient P after the alignment process. If it is determined that the current alignment process of the medical image processing device 300 is not being performed correctly or if it is determined that it is necessary to adjust the position of the patient P, the user can instruct the medical image processing device 300 to perform the alignment process again or to adjust the position of the patient P. Thereby, the treatment system 3 including the medical image processing device 300 can perform more effective radiation treatment like the treatment system 1 including the medical image processing device 100 and the treatment system 2 including the medical image processing device 200.

As described above, even in the medical image processing device 300, as in the medical image processing device 100 and the medical image processing device 200, the first image acquirer 110 acquires a first fluoroscopic image of the patient P photographed before treatment and parameters indicating the position and orientation when the first fluoroscopic image is captured and the second image acquirer 120 acquires a second fluoroscopic image of the patient P photographed immediately before treatment starts and parameters indicating the position and orientation when the second fluoroscopic image is captured. Furthermore, in the medical image processing device 300, as in the medical image processing device 100 and the medical image processing device 200, the treatment error acquirer 130 acquires a treatment error in radiation treatment not appearing in the treatment planning phase. Also, in the medical image processing device 300, as in the medical image processing device 100 and the medical image processing device 200, the difference calculator 140 changes the parameters indicating the position and orientation of the second fluoroscopic image after the alignment process on the basis of the treatment error (by applying a virtual perturbation) and calculates a difference image. Subsequently, in the medical image processing device 300, as in the medical image processing device 100 and the medical image processing device 200, the differential statistical quantity calculator 150 calculates the differential statistical quantity on the basis of the difference image. Also, in the medical image processing device 300, as in the medical image processing device 200, the determiner 260 determines whether or not it is necessary to adjust the position of the patient P on the basis of the differential statistical quantity calculated by the differential statistical quantity calculator 150. Also, in the medical image processing device 300, the patient table controller 380 generates a movement control signal corresponding to the result of the alignment process performed by the position error calculator 370 and moves the patient table 12 to the treatment device 10. Also, in the medical image processing device 300, as in the medical image processing device 100 and the medical image processing device 200, the presentation data processor 390 generates presentation data (the display screen DS) for presenting information such as a calculated differential statistical quantity to the user and causes the display device D to display the presentation data (the display screen DS). Thereby, in the treatment system 3 including the medical image processing device 300, as in the treatment system 1 including the medical image processing device 100 and the treatment system 2 including the medical image processing device 200, the user can determine whether or not the current alignment process of the medical image processing device 300 is being performed correctly with reference to the presentation data (the display screen DS) displayed on the display device D. Furthermore, in the treatment system 3 including the medical image processing device 300, the user can also determine whether or not to adjust the position of the patient P with reference to a determination result of the determiner 260 presented as the presentation data.

As described above, the medical image processing device 300 further includes the position error calculator 370 configured to perform the alignment process with respect to the medical image processing device 200 of the second embodiment; and the patient table controller 380 configured to generate a movement control signal for controlling movement of the patient table 12 provided in the treatment device 10 for performing the radiation treatment on the basis of a result of the alignment process and cause the patient table 12 to move, wherein the difference calculator 140a calculates the difference image to which the perturbation is applied at the position of the patient P shown in the second fluoroscopic image on the basis of the result of the alignment process. Thereby, in the medical image processing device 300, the patient table controller 380 cause the patient table 12 to move to the treatment device 10 on the basis of an amount of movement indicated in a result of performing the alignment process in the position error calculator 370.

As described above, the medical image processing device 300 further includes the presentation data processor 390 configured to generate presentation data for presenting a graph or a numerical value based on at least one of the first fluoroscopic image, the second fluoroscopic image, and the statistical quantity. Thereby, the medical image processing device 300 can present the presentation data generated by the presentation data processor 390 to the user.

A configuration of the second embodiment in which a constituent element, which is a feature of the second embodiment, (the determiner 260) is added to the medical image processing device 100 of the first embodiment and a configuration of the third embodiment in which constituent elements, which are features of the third embodiment, (the position error calculator 370, the patient table controller 380, and the presentation data processor 390) are added to the medical image processing device 200 of the second embodiment have been described. However, the added constituent element in each embodiment may not necessarily be a constituent element that needs to be added. For example, the medical image processing device 300 of the third embodiment may have a configuration in which the determiner 260 added to the medical image processing device 200 of the second embodiment is omitted. In this case, a medical image processing device for implementing the function of each constituent element provided in the medical image processing device is provided.

In each embodiment, a configuration in which the medical image processing device and the treatment device 10 are separate devices has been described. However, the medical image processing device and the treatment device 10 are not limited to configurations that are separate devices and may have a configuration in which the medical image processing device and the treatment device 10 are integrated.

As described above, for example, a medical image processing method to be executed by the medical image processing device 100 includes acquiring, by a computer (a processor or the like), a first fluoroscopic image obtained by photographing the inside of a body of the patient P; acquiring, by the computer, a second fluoroscopic image of the inside of the body of the patient P photographed at a timing different from that of the first fluoroscopic image; acquiring, by the computer, a treatment error occurring when an alignment process of aligning the position of the patient P shown in the second fluoroscopic image with the position of the patient P shown in the first fluoroscopic image is performed on the basis of the first fluoroscopic image and the second fluoroscopic image or a treatment error occurring in radiation treatment; applying, by the computer, a virtual perturbation to the position of the patient P shown in the second fluoroscopic image on the basis of the treatment error and calculating a difference image between the second fluoroscopic image to which the perturbation is applied and the first fluoroscopic image; and calculating, by the computer, the statistical quantity of the difference between the first fluoroscopic image and the second fluoroscopic image to which the perturbation is applied on the basis of the difference image.

As described above, for example, there is provided a program, executed by the medical image processing device 100, for causing a computer (a processor or the like) to: acquire a first fluoroscopic image obtained by photographing the inside of a body of the patient P; acquire a second fluoroscopic image of the inside of the body of the patient P photographed at a timing different from that of the first fluoroscopic image; acquire a treatment error occurring when an alignment process of aligning the position of the patient P shown in the second fluoroscopic image with the position of the patient P shown in the first fluoroscopic image is performed on the basis of the first fluoroscopic image and the second fluoroscopic image or a treatment error occurring in radiation treatment; apply a virtual perturbation to the position of the patient P shown in the second fluoroscopic image on the basis of the treatment error and calculate the difference image between the second fluoroscopic image to which the perturbation is applied and the first fluoroscopic image; and calculate the statistical quantity of the difference between the first fluoroscopic image and the second fluoroscopic image to which the perturbation is applied on the basis of the difference image.

As described above, for example, there is provided a computer-readable non-transitory storage medium storing a program, executed by the medical image processing device 100, for causing a computer (a processor or the like) to: acquire a first fluoroscopic image obtained by photographing the inside of a body of the patient P; acquire a second fluoroscopic image of the inside of the body of the patient P photographed at a timing different from that of the first fluoroscopic image; acquire a treatment error occurring when an alignment process of aligning the position of the patient P shown in the second fluoroscopic image with the position of the patient P shown in the first fluoroscopic image is performed on the basis of the first fluoroscopic image and the second fluoroscopic image or a treatment error occurring in radiation treatment; apply a virtual perturbation to the position of the patient P shown in the second fluoroscopic image on the basis of the treatment error and calculate a difference image between the second fluoroscopic image to which the perturbation is applied and the first fluoroscopic image; and calculate the statistical quantity of the difference between the first fluoroscopic image and the second fluoroscopic image to which the perturbation is applied on the basis of the difference image.

According to at least one embodiment described above, there are provided a first image acquirer (110) configured to acquire a first fluoroscopic image obtained by photographing the inside of a body of a patient (P); a second image acquirer (120) configured to acquire a second fluoroscopic image of the inside of the body of the patient (P) photographed at a timing different from that of the first fluoroscopic image; a treatment error acquirer (130) configured to acquire a treatment error occurring when an alignment process of aligning the position of the patient (P) shown in the second fluoroscopic image with the position of the patient (P) shown in the first fluoroscopic image is performed on the basis of the first fluoroscopic image and the second fluoroscopic image or a treatment error occurring in treatment (radiation treatment); a difference calculator (140) configured to apply a virtual perturbation to the position of the patient (P) shown in the second fluoroscopic image on the basis of the treatment error and calculate a difference image between the second fluoroscopic image (the 2P^{th} fluoroscopic image) to which the perturbation is applied and the first fluoroscopic image; and a differential statistical quantity calculator (150) configured to calculate the statistical quantity of the difference (a differential statistical quantity) between the first fluoroscopic image and the second fluoroscopic image (the 2P^{th} fluoroscopic image) to which the perturbation is applied on the basis of the difference image, whereby a practitioner (user) of radiation treatment such as a doctor can quantitatively confirm a result of alignment of the patient (P) in an image collation process between CT images (the first fluoroscopic image and the second fluoroscopic image) captured at the time of treatment planning and in a treatment phase.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image processing device comprising:
a first image acquirer configured to acquire a first fluoroscopic image obtained by photographing the inside of a body of a patient;
a second image acquirer configured to acquire a second fluoroscopic image of the inside of the body of the patient photographed at a timing different from that of the first fluoroscopic image;
a treatment error acquirer configured to acquire a treatment error occurring when an alignment process of aligning a position of the patient shown in the second fluoroscopic image with a position of the patient shown in the first fluoroscopic image is performed based on the first fluoroscopic image and the second fluoroscopic image or a treatment error occurring in treatment;
a difference calculator configured to apply a virtual perturbation to the position of the patient shown in the second fluoroscopic image based on the treatment error and calculate a difference image between the second fluoroscopic image to which the perturbation is applied and the first fluoroscopic image; and
a differential statistical quantity calculator configured to calculate a statistical quantity of a difference between the first fluoroscopic image and the second fluoroscopic image to which the perturbation is applied based on the difference image.

2. The medical image processing device according to claim 1,
wherein the difference calculator applies perturbations of a plurality of phases to the position of the patient shown in the second fluoroscopic image and calculates the difference image for each of the applied perturbations, and
wherein the differential statistical quantity calculator calculates the statistical quantity corresponding to each of the perturbations based on each difference image.

3. The medical image processing device according to claim 1, further comprising a determiner configured to determine a result of the alignment process based on the statistical quantity.

4. The medical image processing device according to claim 1, further comprising:
a position error calculator configured to perform the alignment process; and
a patient table controller configured to generate a movement control signal for controlling movement of a patient table provided in a treatment device for performing the treatment based on a result of the alignment process and cause the patient table to move,
wherein the difference calculator calculates the difference image to which the perturbation is applied at the position of the patient shown in the second fluoroscopic image based on the result of the alignment process.

5. The medical image processing device according to claim 1, further comprising a presentation data processor configured to generate presentation data for presenting a graph or a numerical value based on at least one of the first fluoroscopic image, the second fluoroscopic image, and the statistical quantity.

6. The medical image processing device according to claim 1, wherein the difference image is an image indicating a path of radiation with which the patient is irradiated in the treatment.

7. The medical image processing device according to claim 1, wherein the treatment error is a movement error assumed to occur when the patient table provided in a treatment device is moved for the treatment.

8. The medical image processing device according to claim 1, wherein the statistical quantity is calculated based on the difference image of a range based on an irradiation direction in which the patient is irradiated with radiation in the treatment and a spatial positional relationship between an irradiator configured to radiate the radiation and a region of a tumor located inside of the body of the patient.

9. A treatment system comprising:
the medical image processing device according to claim 1; and
a treatment device including
an irradiator configured to irradiate the patient with radiation;
an imaging device configured to capture the first fluoroscopic image and the second fluoroscopic image; and
a patient table on which the patient is placed and fixed.

10. A medical image processing method comprising:
acquiring, by a computer, a first fluoroscopic image obtained by photographing the inside of a body of a patient;
acquiring, by the computer, a second fluoroscopic image of the inside of the body of the patient photographed at a timing different from that of the first fluoroscopic image;
acquiring, by the computer, a treatment error occurring when an alignment process of aligning a position of the patient shown in the second fluoroscopic image with a position of the patient shown in the first fluoroscopic image is performed based on the first fluoroscopic image and the second fluoroscopic image or a treatment error occurring in treatment;
applying, by the computer, a virtual perturbation to the position of the patient shown in the second fluoroscopic image based on the treatment error and calculating a difference image between the second fluoroscopic image to which the perturbation is applied and the first fluoroscopic image; and
calculating, by the computer, a statistical quantity of a difference between the first fluoroscopic image and the second fluoroscopic image to which the perturbation is applied based on the difference image.

11. A non-transitory computer-readable storage medium storing a program for causing a computer to:
acquire a first fluoroscopic image obtained by photographing the inside of a body of a patient;
acquire a second fluoroscopic image of the inside of the body of the patient photographed at a timing different from that of the first fluoroscopic image;
acquire a treatment error occurring when an alignment process of aligning a position of the patient shown in the second fluoroscopic image with a position of the patient shown in the first fluoroscopic image is performed based on the first fluoroscopic image and the second fluoroscopic image or a treatment error occurring in treatment;
apply a virtual perturbation to the position of the patient shown in the second fluoroscopic image based on the treatment error and calculate a difference image between the second fluoroscopic image to which the perturbation is applied and the first fluoroscopic image; and
calculate a statistical quantity of a difference between the first fluoroscopic image and the second fluoroscopic image to which the perturbation is applied based on the difference image.
